(19) United States
(12) Patent Application Publication
(10) Pub. No.: US 2015/0185150 A1
(43) Pub. Date: Jul. 2, 2015

(54) METHOD FOR NORMALIZING THE LUMINESCENCE EMITTED BY A MEASURING MEDIUM

(71) Applicants: Gerard Mathis, Bagnols Sur Ceze (FR); Herve Bazin, Villeneuve Les Avignon (FR)

(72) Inventors: Gerard Mathis, Bagnols Sur Ceze (FR); Herve Bazin, Villeneuve Les Avignon (FR)

(73) Assignee: CISBIO BIOASSAYS, Codolet (FR)

(21) Appl. No.: 14/380,259

(22) PCT Filed: Feb. 22, 2012
(86) PCT No.: PCT/FR2012/050370

§ 371 Date: Jan. 12, 2015

(51) Int. Cl. G01N 21/64; G01N 33/542; G01N 33/58

(52) U.S. Cl. CPC G01N 21/6428; G01N 21/6408; G01N 33/582; G01N 33/542; G01N 2021/6439; G01N 2021/6432; G01N 2021/6413; G01N 2201/06113; G06F 19/10

(57) ABSTRACT

The subject matter of the invention is a method for measuring the luminescence of a long-life fluorescent compound present in a measuring medium, said medium containing a biological sample, characterized in that it comprises the following steps:
a) introduction of a long-life fluorescent compound into the measuring medium,
b) introduction, into the measuring medium, of a fluorescent marking agent, the absorption spectrum of which allows the excitation thereof at the same wavelength as that used to excite the long-life fluorescent compound, wherein said emission spectrum allows the measurement of the luminescence thereof at the same wavelength as that used to measure the luminescence of the long-life fluorescent compound,
c) excitation of the measuring medium with a wavelength corresponding to an absorption peak of the long-life fluorescent compound,
d) measurement of the luminescence emitted by the measuring medium immediately after excitation of said medium, mainly corresponding to the luminescence of the marking agent, and for a period of 5 ns to 45 μs, at a wavelength correspondant to an emission peak of the long-life fluorescent compound,
e) time-resolved measurement of the luminescence emitted by the measuring medium at the same wavelength as that used in step d), after a delay of 20 to 200 μs following the excitation of the measuring medium and for a period of 200 to 1000 μs, this luminescence mainly corresponding to that of the long-life fluorescent compound,
f) calculation of a normalized luminescence signal corresponding to the ratio: (signal obtained in step e)/(signal obtained in step d).

### SUBJECT OF THE INVENTION

The invention relates to an improved process for normalizing the luminescence emitted by a measuring medium with respect to the amount of cells contained in this medium. The invention also relates to a reagent kit intended for implementing this process.

### PRIOR ART

Fluorescent compounds constitute a tool of choice for research in biology insofar as they make it possible, in combination with the equipment capable of detecting their luminescence, to visualize biological processes at the cellular, or even molecular, level by microscopy or else by simple measurement of their luminescence using a fluorimeter. These compounds can be used as they are for coloring certain cell or tissue compartments, or else can be conjugated to vectors which will transport them to a specific site. Such vectors may, for example, be antibodies specific for a cell component (such as a protein) that it is desired to label with a fluorescent compound, or else any compound which has a specificity for a cell compartment or component.

The FRET (acronym of “Fluorescence Resonance Energy Transfer”) phenomenon is widely used in biology, in particular for studying molecular interactions. It is based on the use of a fluorescent donor compound (for example a rare earth cryptate or complex) and of an optionally fluorescent acceptor compound, each of these compounds being coupled to a biological molecule. When a biological phenomenon causes these molecules to become close to one another, and the donor compound is excited, energy transfer takes place between the donor and the acceptor and will result in a variation in the luminescence emitted by the reaction medium, in particular an increase in the signal emitted by the acceptor compound and a decrease in the luminescence of the donor. This luminescence can be measured using a fluorimeter adjusted to the emission wavelengths of the fluorescent compounds, and the resulting FRET signal, usually consisting of the ratio of the signal of the acceptor to that of the donor, thus makes it possible to monitor the evolution of the phenomenon observed. The use of this technique with rare earth chelates or cryptates, which has in particular been developed by G. Mathis et al. (“Homogeneous time resolved fluorescence energy transfer using rare earth cryptates as a tool for probing molecular interactions in biology”, Spectrochimica Acta Part A 57 (2001) 2197-2211) has many advantages which have already enabled several applications in the field of in vitro diagnosis and in that of high-throughput screening in the pharmaceutical industry. Several companies market reagents for carrying out this approach in order to study biological processes; for example, the applicant provides various reagents, including rare earth cryptates, for studying particular biological phenomena (detection of enzymatic activity, assaying of second messengers, etc).

Fluorescent rare earth chelates or cryptates, in particular europium or terbium cryptates or chelates, have a lifetime of about one millisecond, which makes it possible to measure the FRET signal emitted by the measuring medium after a delay following the excitation of the donor compound. This time-resolved FRET (TR-FRET) measuring technique makes it possible to dispense with the parasitic luminescence emitted by the measuring medium immediately after excitation thereof and is therefore particularly advantageous when the measuring medium comprises biological material rich in compounds, in particular protein compounds, capable of producing this type of interference.

While the TR-FRET technique has for a long time been used on relatively well-controlled biological systems, such as a given enzyme and its substrate, two proteins which interact with one another, or else the assaying of an analyte in blood plasma or serum, it has recently been applied to much more complex environments, and in particular cells, cell lysates or tissue explants. One of the problems of the in vitro tests carried out on this type of material lies in the difficulty in controlling the number of cells present in the measuring medium, making the comparison of the results obtained in various tests all the more difficult. This technical problem is particularly critical when it is desired to apply the TR-FRET technique to the clinical analysis of tissue samples characterized by a large disparity in cell content.

Techniques for evaluation or else for normalization with respect to the amount of cells present in the measuring medium are available: it is first of all possible to introduce a known number of cells into the measuring medium by distribution of a homogeneous suspension of the cells. Cell counting techniques are also used, but they are relatively fastidious and not very suitable for the analysis of a large number of samples (microscopy, FACS). Another approach consists in evaluating the amount of certain “ubiquitous” proteins, the presence of which indirectly reflects the amount of cells present in the measuring medium (GAPDH, actin). Finally, methods based on the use of DNA-intercalating fluorescent agents are also used: these agents have the particularity of being luminescent at different wavelengths depending on whether they are complexed with DNA; the calculation of the ratio of signals measured at these two wavelengths makes it possible to evaluate the amount of DNA present in the medium. Examples of such intercalating agents are the Hoechst 33258, Hoechst 33342, Hoechst 34580 or DAPI products.

Jensen et al (J Biomol Screen. 2010 Oct. 15(9):1071-81) have, for example, described a technique for studying the membrane potentials of mitochondria, based on the use of fluorescent compounds (DASPEI and TMRM), and including normalization of the fluorescence of these compounds via that of the Hoechst 33258 product. The authors nevertheless had to use no less than six different filters in order to excite and measure the emission of the three fluorescent compounds.

There is today no satisfactory means for studying biological phenomena by the TR-FRET technique on tissue extracts, owing to the variable amounts of cells from one measuring medium to another. The available techniques are fastidious, introduce risks of error and are not suitable for the rapid analysis of several tens or hundreds of samples.

It would therefore be particularly advantageous to be able to measure, by TR-FRET, the luminescence emitted by a medium containing an indeterminate amount of cells, and to be able to calculate a TR-FRET signal taking into account this variable parameter.

### DESCRIPTION OF THE INVENTION

The inventors have developed a process for measuring the luminescence emitted by a measuring medium containing cells, in which the luminescence signal is normalized, i.e. it takes into account the number of cells in the measuring medium.

In its general aspect, the process of the invention consists in using a cell-labeling agent enabling the correction of the luminescence signal so as to take into account the amount of cells present in said medium.

The cell-labeling agent may be:
an intercalating agent capable of labeling the cells at the DNA level, or else
an agent capable of labeling the cells at the level of structural elements of the cell, the amount of which is relatively constant from one cell to another (for example: lipid membranes, total proteins, organelles, ubiquitous enzymes), or else
an agent capable of labeling the cells at the level of a protein which is artificially expressed by the cell (GAPDH, actin, GST, etc) and similarly expressed in all the cells of the sample, or else
a fluorescent exogenous protein expressed similarly in all the cells of the sample.

This process is optimized so as to be carried out in a small number of steps, which has the advantage of limiting the risk of errors and also of envisioning its use in a high-throughput screening test approach. The invention also relates to a kit containing the reagents essential for carrying out this process.

The process of the invention consists of a process for measuring the luminescence of a long-life fluorescent compound present in a measuring medium, said medium containing a biological sample, characterized in that it comprises the following steps:
a) introduction of a long-life fluorescent compound into the measuring medium,
b) introduction, into the measuring medium, of a fluorescent labeling agent, the absorption spectrum of which allows the excitation thereof at the same wavelength as that used to excite the long-life fluorescent compound, and the emission spectrum of which allows the measurement of the luminescence thereof at the same wavelength as that used to measure the luminescence of the long-life fluorescent compound,
c) excitation of the measuring medium at a wavelength corresponding to an absorption peak of the long-life fluorescent compound,
d) measurement of the luminescence emitted by the measuring medium immediately after the excitation of said medium, mainly corresponding to the luminescence of the labeling agent, and for a period of 5 ns to 45 μs, at a wavelength corresponding to an emission peak of the long-life fluorescent compound,
e) time-resolved measurement of the luminescence emitted by the measuring medium at the same wavelength as that used in step d), after a delay of 20 to 200 μs following the excitation of the measuring medium and for a period of 200 to 1000 μs, this luminescence mainly corresponding to that of the long-life fluorescent compound,
f) calculation of a normalized luminescence signal corresponding to the ratio: (signal obtained in step e)/(signal obtained in step d).

In one preferred implementation, the luminescence measured is that subsequent to a TR-FRET phenomenon in the measuring medium, i.e. a transfer of energy between a donor fluorescent compound and an acceptor fluorescent compound. In this implementation, the long-life fluorescent compound acts as the energy donor and the previous process then comprises the following steps:
a) introduction of a long-life fluorescent compound into the measuring medium,
b) introduction, into the measuring medium, of an acceptor fluorescent compound, the absorption spectrum of which is compatible with the emission spectrum of the long-life fluorescent compound, the long-life fluorescent compound and the acceptor fluorescent compound being FRET partners,
c) introduction, into the measuring medium, of a fluorescent labeling agent, the absorption spectrum of which allows the excitation thereof at the same wavelength as that used to excite the long-life fluorescent compound, and the emission spectrum of which allows the measurement of the luminescence thereof at the same wavelength as that used to measure the luminescence of the long-life fluorescent compound,
d) excitation of the measuring medium at a wavelength corresponding to an absorption peak of the long-life fluorescent compound,
e) measurement of the luminescence emitted by the measuring medium immediately after the excitation of said medium, mainly corresponding to the luminescence of the labeling agent, and for a period of 5 ns to 45 μs, at a wavelength corresponding to an emission peak of the long-life fluorescent compound,
f) optionally, time-resolved measurement of the luminescence emitted by the measuring medium at the same wavelength as that used in step e), after a delay of 20 to 200 μs following the excitation of the measuring medium and for a period of 200 to 1000 μs,
g) time-resolved measurement of the luminescence emitted by the measuring medium after a delay of 20 to 200 μs after the excitation of the measuring medium and for a period of 200 to 600 μs, at the emission wavelength of the acceptor compound,
h) determination of a normalized TR-FRET signal comprising the calculation of the ratio: (signal obtained in step g)/[(optionally signal obtained in step f)×(signal obtained in step e)].

In this implementation, step f) is therefore optional: it is possible to dispense with the measurement of the luminescence of the long-life fluorescent compound.

In one preferred implementation according to which the luminescence measured is that subsequent to a TR-FRET phenomenon in the measuring medium, it is possible to measure the luminescence of the labeling agent and that of the acceptor fluorescent compound at the same wavelength. In this case, of course, the luminescence of the labeling agent and that of the long-life fluorescent compound are no longer measured at the same wavelength, and the process is modified in the following way:
in step c), the labeling agent is a fluorescent labeling agent, the absorption spectrum of which allows the excitation thereof at the same wavelength as that used to excite the long-life fluorescent compound, and the emission spectrum of which allows the measurement of the luminescence thereof at the same wavelength as that used to measure the luminescence of the acceptor fluorescent compound,
in step e), the luminescence is measured at a wavelength corresponding to an emission peak of the acceptor fluorescent compound,
in step f), if it is present, the luminescence is measured at a wavelength corresponding to an emission peak of the long-life fluorescent compound.

In one particular implementation, the process according to the invention also comprises a second step of excitation of the measuring medium at a wavelength corresponding to an absorption peak of said long-life fluorescent compound, this second excitation being carried out immediately before the step of time-resolved measurement of the luminescence.

In another particular implementation and when the biological sample consists of tissue or of intact cells, the process according to the invention also comprises a step of washing before the first excitation, i.e. between steps b) and c) of the process or steps c) and d) of the preferred implementation thereof.

### Measuring Medium, Biological Material

The process according to the invention is carried out on a measuring medium containing a biological sample. The term “biological sample” is intended to mean a tissue sample (in particular a tumor sample) or else a sample of cells from a cell culture. One of the advantages of this process lies in the fact that it makes it possible to normalize the TR-FRET signal with respect to the amount of cells present in this measuring medium.

The cells present in the measuring medium may be isolated cells, for example cells obtained by cell culture. They may be in suspension or else may adhere to the container of the measuring medium. These cells may also be from a tissue extract. In this case, the biological sample preferably consists of histological sections, in particular frozen histological sections. The preparation of histological sections falls within the general knowledge of those skilled in the art: they are prepared, for example, using a cryotome or an ultramicrotome on tissues embedded in paraffin or epoxy resins. Preferably, a cryotome is used with non-fixed frozen tissues, in order to obtain frozen sections and to avoid denaturing proteins.

The process of the invention is particularly advantageous when it is carried out on a tissue extract owing to the variability of the amount of biological material present in this case.

In any event, the cells may be intact, or else have been the subject of treatment aimed at homogenizing said sample in cell lysate form, before or after the introduction of the fluorescent compounds into the measuring medium. Such a treatment may be mechanical, resulting in a cell lysate. The latter implementation is preferred when the process is performed on tissue extracts, since it provides greater reliability of the measurement of the luminescence emitted, which must be measured using the most homogeneous measuring medium possible. The mechanical treatment in question can be chosen from: the application of ultrasound, freezing/thawing cycles, the use of mechanical mills, optionally together with the use of hypotonic lysis buffer or of lysis buffer containing detergents, such as the RIPA buffer. When such a treatment is performed, and depending on the subject of the study by those skilled in the art, the latter will take care to use controlled treatment conditions. In particular, if the process is carried out in the context of the study of subcellular structures or else of protein aggregates or oligomers, an excessively fierce mechanical treatment could degrade these structures or oligomers.

An example of controlled mechanical treatment consists in applying ultrasound (sonication) to the measuring medium at a frequency of between 19 800 and 20 500 kHz, at a power of 80 W and for a period of 3 to 10 seconds, and preferably for 4 to 6 seconds. The samples are preferably kept in an ice bath during the treatment in order to limit heating thereof. The inventors have discovered that, surprisingly, this treatment does not significantly disrupt the antigen-antibody bonds or the protein oligomers, in particular the dimers present in the measuring medium.

Those skilled in the art can easily adjust these chemical or mechanical treatment parameters according to the material studied.

### Long-Life Fluorescent Compounds

One of the essential characteristics of the invention is the use of long-life fluorescent compounds. Such compounds are known and are usually fluorescent metallic complexes, having a lifetime greater than 1 μs, and preferably greater than 100 μs.

Generally, the term “fluorescent metallic complex” is intended to mean a compound consisting of a lanthanide or of ruthenium and of a polydentate complexing agent, i.e. comprising at least 2, and preferably between 2 and 9, electron donor heteroatoms, such as N, O or S, these atoms forming coordination bonds with the lanthanide or the ruthenium. Preferably, the fluorescent metallic complex comprises one or more chromophores consisting of aromatic structures; preferably, these aromatic structures comprise 1, 2 or 3 heteroatoms chosen from N and O, which act as lanthanide or ruthenium coordination atoms.

A fluorescent metallic complex suitable for the purposes of the invention must be stable in terms of association/dissociation of the complexing agent and of the rare earth element, and preferably its formation constant (Kf) should be greater than 10¹⁰ M⁻¹.

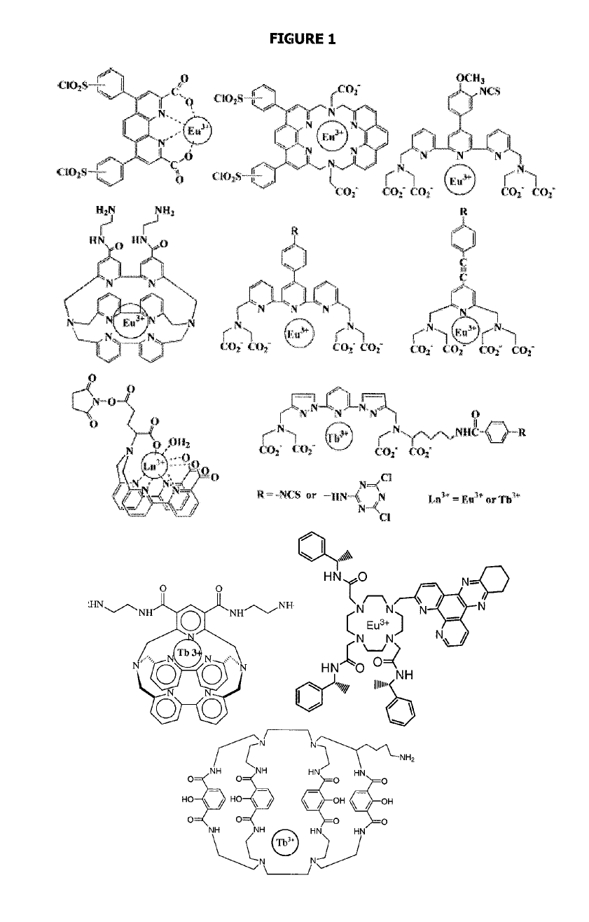

Many complexing agents have been described and are known to those skilled in the art: by way of examples of complexing agents, mention may be made of the following compounds: EDTA, DTPA, TTHA, DOTA, NTA, HDTA, DTPP, EDTP, HDTP, NTP, DOTP, DO3A and DOTAGA. Terbium and europium chelates and crypates have been described and several are commercially available. Examples of long-life fluorescent compounds are given in FIG. 1, and also hereinafter:
Lanthanide cryptates comprising one or more pyridine units. Such rare earth cryptates are described, for example, in patents EP 0 180 492, EP 0 321 353 and EP 0 601 113 and in international application WO 01/96877. Terbium (Tb3+) cryptates and europium (Eu3+) cryptates are particularly suitable for the purposes of the present invention. Lanthanide cryptates are sold by the company Cisbio Bioassays. By way of nonlimiting example, mention may be made of the europium cryptates having the formulae below (which can be coupled to the compound to be labeled by a reactive group, in this case for example an NH₂ group):

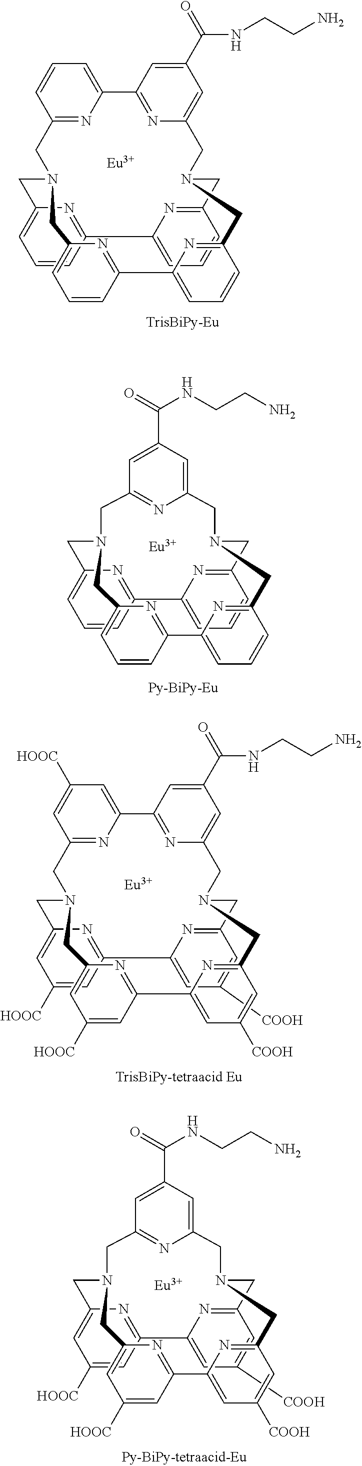

The lanthanide chelates described in particular in patents U.S. Pat. No. 4,761,481, U.S. Pat. No. 5,032,677, U.S. Pat. No. 5,055,578, U.S. Pat. No. 5,106,957, U.S. Pat. No. 5,116,989, U.S. Pat. No. 4,761,481, U.S. Pat. No. 4,801,722, U.S. Pat. No. 4,794,191, U.S. Pat. No. 4,637,988, U.S. Pat. No. 4,670,572, U.S. Pat. No. 4,837,169 and U.S. Pat. No. 4,859,777. Patents EP 0 403 593, U.S. Pat. No. 5,324,825, U.S. Pat. No. 5,202,423 and U.S. Pat. No. 5,316,909 describe chelates composed of a nonadentate ligand such as terpyridine. Lanthanide chelates are sold by the company PerkinElmer.
Lanthanide complexes consisting of a chelating agent, such as tetraazacyclododecane, substituted with a chromophore comprising aromatic rings, such as those described by R. Poole et al., in Biomol. Chem, 2005, 3, 1013-1024 “Synthesis and characterisation of highly emissive and kinetically stable lanthanide complexes suitable for usage in cellulo”, can also be used. The complexes described in application WO 2009/10580 can also be used.
The lanthanide cryptates described in patents EP 1 154 991 and EP 1 154 990 are also usable.
The terbium cryptate having the formula below (which can be coupled to a compound to be labeled via a reactive group, in this case for example an NH₂ group):

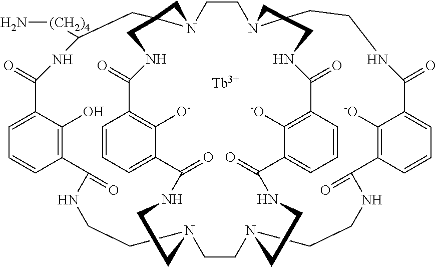

and the synthesis of which is described in international application WO 2008/063721 (compound 6a, page 89).
The terbium cryptate Lumi4-Tb from the company Lumiphore, sold by Cisbio Bioassays.
The quantum dye from the company Research Organics, having the formula below (which can be coupled to the compound to be labeled via a reactive group, in this case NCS):

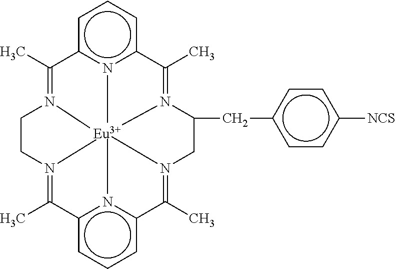

Ruthenium chelates, in particular complexes consisting of a ruthenium ion and of several bipyridines, for instance ruthenium(II) tris(2,2′-bipyridine).
The terbium chelate DTPA-cs124 Tb, sold by the company Life Technologies, having the formula below (which can be coupled to the compound to be labeled via a reactive group R) and the synthesis of which is described in American patent U.S. Pat. No. 5,622,821.

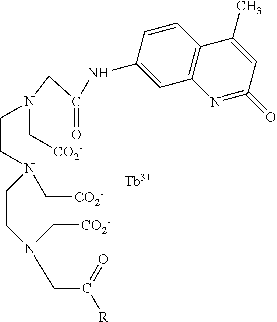

The terbium chelate having the formula below and which is described by Latva et al. (Journal of Luminescence 75: 149-169):

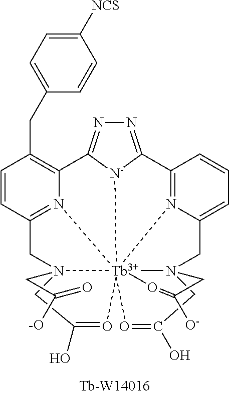

Particularly advantageously, the long-life fluorescent compound is a fluorescent metallic complex chosen from: a europium cryptate; a europium chelate; a terbium chelate; a terbium cryptate; a ruthenium chelate; and a quantum dye; the europium and terbium chelates and cryptates being particularly preferred.

Dysprosium (Dy3+), samarium (Sm3+), neodymium (Nd3+), ytterbium (Yb3+) or erbium (Er3+) complexes are also rare earth complexes that are suitable for the purposes of the invention.

### Acceptor Compounds

In the particular implementation of the invention according to which the luminescence emitted by the measuring medium results from a TR-FRET phenomenon, an energy acceptor compound compatible with the long-life fluorescent compound is introduced into this medium. The selection of the donor/acceptor fluorophore pair for obtaining a FRET signal is within the scope of those skilled in the art. Generally, in order for the FRET to take place, the excitation spectrum of the acceptor must at least partly overlap the emission spectrum of the donor compound, and the transition dipoles of the donor and acceptor compounds must be parallel. Donor-acceptor pairs that can be used for studying FRET phenomena are in particular described in the book by Joseph R. Lakowicz (Principles of fluorescence spectroscopy, 2^{nd} edition 338), to which those skilled in the art may refer.

The acceptor fluorescent compounds can be chosen from the following group: allophycocyanins, in particular those known under the trade name XL665; luminescent organic molecules, such as rhodamines, cyanines (for instance Cy5), squaraines, coumarins, proflavines, acridines, fluoresceins, boron-dipyrromethane derivatives (sold under the name “Bodipy”), fluorophores known under the name “Atto”, fluorophores known under the name “DY”, compounds known under the name “Alexa”, and nitrobenzoxadiazole. Advantageously, the acceptor fluorescent compounds are chosen from allophycocyanins, rhodamines, cyanines, squaraines, coumarins, proflavines, acridines, fluoresceins, boron-dipyrromethane derivatives and nitrobenzoxadiazole.

The expressions “cyanines” and “rhodamines” should be respectively understood as “cyanine derivatives” and “rhodamine derivatives”. Those skilled in the art are aware of these various commercially available fluorophores.

The “Alexa” compounds are sold by the company Invitrogen; the “Atto” compounds are sold by the company Attotec; the “DY” compounds are sold by the company Dyomics; the “CY” compounds are sold by the company Amersham Biosciences; the other compounds are sold by various suppliers of chemical reagents, such as the companies Sigma, Aldrich or Acros.

The following fluorescent proteins can also be used as acceptor fluorescent compound: cyan fluorescent proteins (AmCyan1, Midori-Ishi Cyan, mTFP1), green fluorescent proteins (EGFP, AcGFP, TurboGFP, Emerald, Azami Green, ZsGreen), yellow fluorescent proteins (EYFP, Topaz, Venus, mCitrine, YPet, PhiYFP, ZsYellow1, mBanana), orange and red fluorescent proteins (Orange kusibari, mOrange, tdtomato, DsRed, DsRed2, DsRed-Express, DsRed-Monomer, mTangerine, AsRed2, mRFP1, JRed, mCherry, mStrawberry, HcRed1, mRaspberry, HcRed-Tandem, mPlim, AQ143), and proteins which are fluorescent in the far-red range (mKate, mKate2, tdKatushka2).

For the purposes of the invention, the cyanine derivatives are preferred as acceptor fluorescent compound.

The donor and/or acceptor fluorescent compounds are generally each conjugated to a member of a pair of binding partners, depending on the subject studied by those skilled in the art, in accordance with the usual application of the TR-FRET technique to the study of biological phenomena. In particular, these compounds can each be coupled to an antibody, an enzyme substrate, a peptide, a membrane receptor ligand, etc. The nature and the function of these molecules conjugated to the donor and acceptor compounds do not constitute essential technical features of the process according to the invention: they are variable parameters that those skilled in the art will adapt according to the subject of their study, and that will not have any effect on the process for normalizing the FRET signal according to the invention. It is for this reason that these aspects, widely described elsewhere, are not explained here.

### Fluorescent Labeling Agents: Intercalating Agent

In one preferred implementation, the labeling agent used in the process according to the invention is a fluorescent intercalating agent. The expression “intercalating agent” is used here in the broad sense, and denotes in particular an organic compound capable of integrating into the DNA double helix, either by intercalating between the DNA strands, or by binding to the small groove or to the large groove of the DNA double helix. These agents are known and their spectroscopic characteristics, in particular their emission spectra, are different depending on whether or not they are complexed with DNA. These commercially available products thus constitute one of the technical solutions available for quantifying the amount of cells present in a measuring medium.

It is important to underline that the use of such intercalating agents in combination with other fluorophores, in particular terbium earth complexes or chelates which emit at the same wavelength, goes against a technical presupposition that, when several fluorescent compounds are used, it is generally preferable for these compounds to emit at different wavelengths in order to avoid the luminescence of one contaminating the luminescence of the other.

The inventors have determined the experimental conditions which make it possible to avoid contamination of the signal of the intercalating agent by that of the long-life fluorescent compound. In particular, the concentration of the intercalating agent in the measuring medium must be greater than that of the long-life fluorescent compound, preferably by at least 50 to 150 times. Generally, the use of the intercalating agent at a final concentration before complexation of 1 and 10 μM is thus preferred.

Numerous fluorescent intercalating agents have been described. Table 1 hereinafter gives a list of these compounds, and also the information relating to their spectroscopic properties.

TABLE 1

| | Excitation/emission |
|---|---|
| Acridine homodimer | 431/498 |
| Acridine orange | 500/526 (DNA) |
| | 460/650 (RNA) |
| 7-AAD (7-amino-actinomycin | 546/647 |
| D) | |
| ACMA | 419/483 |
| DAPI | 358/461 |
| Dihydroethidium | 518/605 |
| Ethidium bromide | 518/605 |
| Ethidium homodimer-1 | 528/617 |
| (EthD-1) | |
| Ethidium homodimer-2 | 535/624 |
| (EthD-2) | |
| Hexidium iodide | 518/600 |
| Hoechst 33258 (bis- | 352/461 |
| benzimide) | |
| Hoechst 33342 | 350/461 |
| Hoechst 34580 | 392/498 |
| Hoechst S769121 | 355/495 |
| Hydroxystilbamidine | 385/emission varies with |
| | nucleic acid |
| LDS 751 | 543/712 (DNA) |
| | 590/607 (RNA) |
| Nuclear yellow | 355/495 |
| Propidium iodide (PI) | 530/625 |
| OliGreen | 485/535 |
| PicoGreen | 485/535 |
| RiboGreen | 485/535 |
| SybrGreen I | 485/535 |
| YO-YO-1 | 450/550 |
| YO-PRO-1 | 450/550 |

The preferred fluorescent intercalating agents are those which can be used according to the invention with a terbium or europium chelate or cryptate, i.e. which can be excited between 330 and 350 nm, and which emit, after binding to DNA, either at the wavelengths corresponding to the emission peaks of these terbium or europium chelates or cryptates, namely in particular around 588 nm and 620 nm (if a europium chelate or cryptate is used) or around 490 nm, 545 nm, 588 nm or 620 nm (if a terbium chelate or cryptate is used), or at a wavelength corresponding to an emission peak of the acceptor compound, generally between 450 and 650 nm, most commonly between 480 and 600 nm. Preferably, an intercalating agent which is sufficiently lipophilic to enter the cell nucleus will be chosen, in particular when working on whole cells and not on ground cell material. Moreover, since certain intercalating agents are rejected by living cells, it is advisable, when they are used, to introduce them into the measuring medium once the cells have been converted into a cell lysate, for example by sonication. This is not, however, the case of the Hoechst 33342 compound, which can intercalate into the DNA of intact living cells.

Preferably, the fluorescent intercalating agent is chosen from the following group: Hoechst 33258, Hoechst 33342, Hoechst 34580 and DAPI (4′,6′-diamidino-2-phenylindole).

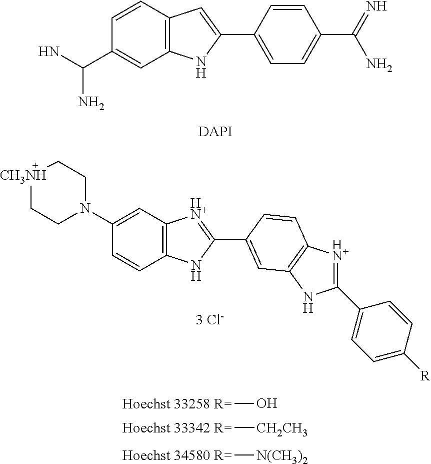

This list is not limiting and those skilled in the art, on the basis of the elements provided by the inventors, will be capable of choosing other agents suitable for the invention.

Even more preferably, the invention is implemented with the Hoechst 33342 intercalating agent, which is ideal when the long-life fluorescent compound is a terbium chelate or cryptate, and which can also be used with a europium chelate or cryptate.

### Other Labeling Agents:

In the process of the invention, the labeling agent is not necessarily an intercalating agent: use may also be made of a fluorescent compound capable of labeling the cells, the absorption spectrum of which allows the excitation thereof at the same wavelength as that used to excite the long-life fluorescent compound, and the emission spectrum of which allows the measurement of the luminescence thereof at the same wavelength as that used to measure the luminescence of the long-life fluorescent compound, or else at that of the acceptor when it is present. Preferably, a labeling agent that is sufficiently lipophilic to enter cells will be chosen, in particular when working on whole cells and not on ground cell material.

The preferred fluorescent intercalating agents are those which can be used according to the invention with a terbium or europium chelate or cryptate, i.e. which can be excited between 330 and 350 nm, and which emit, after binding to DNA, either at the wavelengths corresponding to the emission peaks of these terbium or europium chelates or cryptates, namely in particular around 588 nm and 620 nm (if a europium chelate or cryptate is used) or around 490 nm, 545 nm, 588 nm or 620 nm (if a terbium chelate or cryptate is used), or at a wavelength corresponding to an emission peak of the acceptor compound, generally between 450 and 650 nm, most commonly between 480 and 600 nm.

Examples of such labeling agents are:
fluorescent proteins, such as green fluorescent protein (GFP). When a fluorescent protein is used as labeling agent, the cells of the sample will have been transfected beforehand with an expression vector encoding this fluorescent protein. In this case, the step of the invention consisting in introducing the labeling agent into the measuring medium will correspond, in practice, to the step of introduction of the biological sample into this medium. Expression plasmids encoding these proteins are commercially available and the transfection techniques are known to those skilled in the art. The luminescence of the fluorescent protein will be measured immediately after the excitation of the measuring medium, in a manner similar to the case where the labeling agent is an intercalating agent;
fluorescent compounds labeling mitochondria, such as Mitotracker Orange, Mitotracker Green, or else rhodamine 123. These compounds are commercially available and the protocols for labeling cells with these compounds are also known. They can be used according to the invention with a protocol similar to the case where the labeling agent is an intercalating agent;
fluorescent compounds, such as dansyl chloride or NBD (nitrobenzoxadiazole): these compounds, which are also commercially available, bind to amine functions, in particular of proteins. Y. Uratani et al. describe a protocol for labeling cells with dansyl chloride (Journal of Bacteriology 1982 p. 523-528). Once the cells have been labeled, the invention can be implemented with a protocol similar to the case where the labeling agent is an intercalating agent;
fluorescent compounds which accumulate in lipid membranes, such as filipin: this compound is commercially available and its use for labeling cells is known. It can be used according to the invention with a protocol similar to that of the case where the labeling agent is an intercalating agent;
fluorescent compounds capable of labeling a protein which the cell is made to artificially express: in this implementation, the cells have been transfected beforehand with a plasmid encoding a fusion protein comprising a suicide enzyme, such as the enzymes sold under the name Snaptag, Cliptag, ACPtag, MCPtag or Halotag. This fusion protein can also comprise the GST protein, actin, GAPDH or any other protein readily expressed by a cell. Bringing these cells into contact with a fluorescent substrate of these enzymes (such as a benzylguanine-fluorophore, benzylcytosine-fluorophore or chloroalkane-fluorophore derivative) will cause labeling of the fusion protein. The signal of the fusion protein thus labeled can be measured and used according to the invention with a protocol similar to that used in the case where the labeling agent is an intercalating agent.

### Excitation and Measurement of the Luminescence

The invention constitutes an improvement of the known methods using a long-life fluorescent compound, and optionally also an acceptor fluorescent compound, in that it comprises the use of a fluorescent labeling agent and that it proposes optimizing the excitation of these compounds and measuring their luminescence. The luminescence emitted by the measuring medium is produced conventionally using a fluorimeter which allows the excitation of the medium at a given wavelength, and the measurement of the luminescence emitted after excitation in a time window and at a wavelength which are chosen by the user.

### Excitation of the Long-Life Fluorescent Compound and of the Labeling Agent

One of the originalities of the invention consists in exciting the measuring medium in such a way that not only the donor compound but also the labeling agent are excited simultaneously, or in any event at the same wavelength. For this, the invention is based on the use of long-life fluorescent compounds and of fluorescent labeling agents, the absorption spectra of which allow excitation at the same wavelength.

Since nitrogen laser excitation fluorimeters allow only excitation at 337 nm, long-life donor fluorescent compounds and labeling agents of which the absorption spectra allow excitation at this wavelength are preferred. This condition is met in particular by europium or terbium chelates and cryptates, and also by a certain number of fluorescent intercalating agents listed in Table 1.

When flash lamp fluorimeters are used, the excitation wavelength can be modified by the user according to the long-life fluorescent compounds and to the labeling agents that said user wishes to use. Nevertheless, when these fluorimeters are used, the excitation wavelength will preferably be chosen between 320 and 360 nm, and preferably between 330 and 350 nm.

Finally, in one particular implementation, the measuring medium is excited twice, at the same wavelength: a first time in order to measure the fluorescence of the labeling agent (continuous fluorescence, measured without delay after excitation), and a second time for time-resolved measurement of the fluorescence. This is particularly advantageous when it is desired to modify the gain of the fluorimeter, which may be necessary in particular if the labeling agent signal saturates the measuring channel.

### Measurement of the Luminescence Emitted

Following the excitation of the measuring medium, the latter will emit luminescence corresponding to the excitation of the long-life fluorescent compound and of the fluorescent labeling agent. In the implementation according to which the medium also comprises an acceptor fluorescent compound which is a FRET partner of the long-life fluorescent compound, this acceptor compound will also emit. It is necessary to recall that the measurement of the luminescence at a given wavelength amounts in practice, owing to the technical constraints of fluorimeters, to measuring the luminescence in a more or less wide range centered around the emission wavelength targeted. Typically, when a fluorimeter is adjusted for a measurement at a wavelength L, the luminescence emitted at L nm+/−5, 10 or 20 nm is in practice measured.

The expression “TR-FRET signal” sometimes used is a misuse of language which in fact denotes the luminescence emitted by the measuring medium following the transfer of energy from the donor compound to the acceptor compound. This TR-FRET signal can correspond to the luminescence of the donor compound, the intensity and the lifetime of which will decrease following the energy transfer, or else to that of the acceptor compound, the intensity of which will increase. Most commonly, the two signals are measured and the TR-FRET signal consists of the ratio of the signal of the acceptor to that of the donor.

According to one of the implementations of the invention, the luminescence of the labeling agent and that of the donor fluorescent compound are measured at the same wavelength. This makes it possible to further reduce the time for implementing the process since no change of filter is necessary in order to measure the signal of each of these compounds, and, moreover, to limit the risk of error associated with the modification of the measuring conditions. This is once again made possible by the choice of a long-life fluorescent compound and of a fluorescent labeling agent, the emission spectra of which allow the measurement of luminescence at the same wavelength.

In another implementation, the luminescence of the labeling agent and that of the acceptor fluorescent compound, when it is present, are measured at the same wavelength. Preferably, the long-life fluorescent compound is a europium or terbium cryptate or chelate, the fluorescent labeling agent is an agent the absorption spectrum of which allows excitation at a wavelength of between 330 and 350 nm and which emits a wavelength of between 450 and 650 nm, in particular between 490 and 600 nm, and the luminescence of the acceptor fluorescent compound and that of the fluorescent labeling agent are both measured at a wavelength included in these ranges.

In the embodiment consisting in using terbium chelates or cryptates as long-life fluorescent compound, the labeling agent will be chosen from those of which the absorption spectrum allows excitation at a wavelength of between 330 and 350 nm, and of which the emission spectra allow the measurement of luminescence at a wavelength close to that of the terbium emission peak maxima, namely 490 nm, 545 nm, 588 nm or 620 nm, in particular at the wavelengths of 490+/−10 nm, 545+/−10 nm or 590+/−10 nm. According to this implementation, the luminescence of the long-life fluorescent compound (terbium chelate or cryptate) and that of the labeling agent will thus both be measured at these wavelengths. Preferably, a labeling agent the emission spectrum of which allows measurement of luminescence at 490+/−10 nm, 545+/−10 nm or 590+/−10 nm is chosen.

Similarly, if the long-life fluorescent compound is a europium chelate or cryptate, then the labeling agent will be chosen from those of which the absorption spectrum allows excitation at a wavelength of between 330 and 350 nm, and of which the emission spectra allow the measurement of luminescence at a wavelength close to that of the europium emission peak maxima, namely 580 nm, 588 nm, 620 nm, 650 nm and 684 nm, in particular at the wavelengths of 588 nm+/−10 nm or 620 nm+/−10 nm; the luminescence of the long-life fluorescent compound and that of the labeling agent will both preferably be measured at the wavelengths of 588 nm+/−10 nm or 620 nm+/−10 nm.

The invention advantageously takes advantage of the spectroscopic properties of these compounds in order to discriminate their respective luminescences, even in the case where they are measured at the same wavelength. According to the invention, the luminescence of the long-life fluorescent compound and that of the labeling agent are in fact measured in different time windows: the inventors have discovered that, unexpectedly, the signal of the long-life fluorescent compound does not contaminate the signal of the labeling agent measured immediately after the excitation of the measuring medium.

Thus, and this is one of the most important characteristics of the invention, the luminescence of the labeling agent is measured immediately after the excitation of the measuring medium, whereas the signals of the long-life fluorescent compound and/or the acceptor, when it is present, are measured after a delay following this excitation, and for a given period of time (also called “integration time”), these two parameters being adjustable on the commercially available fluorimeters.

According to the present invention, the expression “measurement immediately after excitation of the measuring medium” is intended to mean an immediate measurement, i.e. without delay or after a very short period of time not exceeding 30 μs.

In particular, the luminescence of the labeling agent is measured immediately after excitation of the measuring medium, and for the shortest possible period of time on the fluorimeter. When the excitation is carried out by a laser, the signal can be measured in a window of 5 ns to 8 μs. On some laser excitation instruments, it is recommended to carry out the measurement in one of the following ranges: 0-8 μs, 2-8 μs, 2-6 μs and 2-4 μs. When the excitation is carried out using a flash lamp, the signal is preferably measured for a little longer, namely for a period of time of 5 μs to 45 μs, preferably of 5 μs to 20 μs.

The luminescence emitted by the measuring medium and corresponding to the signal of the donor compound is measured subsequent to that of the labeling agent, after a delay of 20 to 200 μs, and preferably of 40 to 200 μs following excitation of the measuring medium, and for a period of 200 to 1000 μs, preferably of 400 to 1000 μs. The following time windows, individually, are preferred windows: 50-550 μs, 100-500 μs, 100-300 μs, 200-1200 μs.

The term “time window” is intended to mean the period of time which begins when the luminescence is measured after a delay following light excitation of the measuring medium and ends at the end of the integration time. For example, a “100-500 μs” time window means that the luminescence is measured after a delay of 100 μs after light excitation of the measuring medium, for a period (integration time) of 400 μs.

Finally, the luminescence of the acceptor compound, when it is present, is measured at its emission wavelength, either in the same time window as that used to measure the signal of the long-life fluorescent compound, or in a different window. In the latter case, the luminescence of the acceptor compound will be measured at its emission wavelength after a delay of 20 to 200 μs, preferably of 40 to 200 μs, after excitation of the measuring medium and for a period of 200 to 600 μs, preferably of 400 to 600 μs.

In the case where the measuring medium contains an acceptor compound and a TR-FRET signal is measured, its evolution will allow those skilled in the art to gain a better understanding of the biological system that they are studying, since this signal depends on the proximity of the donor and acceptor fluorescent compounds that they introduced into the measuring medium. The invention does not relate to the various applications as such of the TR-FRET technique to the study of the biological systems that have been described elsewhere, but it nevertheless makes it possible to improve them. As indicated above, when an energy transfer takes place between a donor compound and an acceptor compound, the luminescence of the donor will decrease when the energy transfer increases, and that of the acceptor will increase. Thus, the TR-FRET signal representative of an evolution of the biological system observed may be either that of the donor, or that of the acceptor. It is nevertheless extremely common to measure the two signals and to divide the signal of the acceptor compound by that of the donor compound. Fluorimeters generally include a mode for this type of reading.

In any event, whether the TR-FRET signal is that of the donor, that of the acceptor or else a ratio of the acceptor/donor signals, this signal will in addition, and according to the invention, be corrected by that of the fluorescent labeling agent, this correction making it possible to normalize the TR-FRET signal in terms of the DNA content of the measuring medium, and therefore in terms of the amount of cells. Preferably, this correction is automatic and is performed by the software which analyzes the signals measured. This software can be integrated into the fluorimeter or else operate on a computer which is separate from said fluorimeter and to which the results of the fluorescence measurement are submitted.

In the case where the format is a TR-FRET format, it is recommended to determine the contribution of the long-life fluorescent compound to the luminescence emitted, in a time-resolved manner, at the emission wavelength of the acceptor fluorescent compound, by establishing a calibration range which will make it possible to determine the relationship between, on the one hand, the signal of the long-life compound emitted, in a time-resolved manner, at the emission wavelength of this compound, and, on the other hand, the signal emitted by this same compound in a time-resolved manner, but at the emission wavelength of the acceptor compound, this being in the absence of FRET—or in the absence of acceptor. This calibration range makes it possible to determine the coefficients of the linear function which links these two signals, and which makes it possible to calculate the parasitic contribution. This method is illustrated in Example 5.3.2.

Finally, the method according to the invention can be further refined in order to take into account the fact that the long-life compound can contribute, in minor fashion, to the signal measured immediately after excitation (signal corresponding predominantly to that of the labeling agent). It is possible to calculate this parasitic contribution by establishing a calibration range that will make it possible to determine the relationship between the signal of the long-life compound measured in time-resolved fashion and that measured by continuous fluorescence (without delay). For this, the signal emitted by a measuring medium containing the long-life fluorescent compound but no labeling agent will be measured at increasing concentrations of long-life fluorescent compound, on the one hand by continuous fluorescence and on the other hand by time-resolved fluorescence. The relationship between the two signals is a linear function which makes it possible to calculate the contribution of the long-life fluorescent compound to the signal emitted by continuous fluorescence, using the value measured in time-resolved manner.

Sequence of Bringing the Fluorescent Compounds into Contact with the Measuring Medium, Washing and Transfer of this Medium from One Container to Another

The process according to the invention can be carried out in various ways, in particular depending on the nature of the biological material present in the measuring medium. When the process according to the invention comprises an additional step of mechanical treatment, such as a step of sonication of the biological material, this step can be carried out either before or after bringing the sample of biological material into contact with the donor and acceptor fluorescent compounds and the labeling agent, and of course always prior to the measurement of the luminescence emitted by the measuring medium.

A first possibility, in the case where the process is carried out on a tissue extract which is subjected to a sonication treatment, consists in bringing the tissue into contact with the fluorescent compounds before the sonication of the sample, according to the following steps:
a) bringing the fluorescent compounds (donor, acceptor, labeling agent) into contact with a tissue sample,
b) washing,
c) sonication,
d) transfer into a microplate (if the tissue was not already in a microplate),
e) reading of the microplate with a fluorimeter (donor signal, acceptor signal, labeling agent signal).

This implementation offers the advantage of comprising a washing step which will limit the background noise and thus increase the sensitivity of the measurement. It is preferred when the biological system studied does not make it possible to measure significant variations in TR-FRET signal. This protocol has been implemented in Examples 3.2, 5.3 and 6.3.

A second possibility, in the case where the process is carried out on a tissue extract which has been subjected to a sonication treatment, consists in bringing the tissue into contact with the fluorescent compounds after having carried out the sonication of the sample, according to the following steps:
a) sonication of the tissue sample,
b) transfer into a microplate (if the tissue was not already in a microplate),
c) addition of the fluorescent compounds (donor, acceptor, labeling agent) to the measuring medium,
d) reading of the microplate with a fluorimeter (donor signal, acceptor signal, labeling agent signal).

This implementation does not comprise a washing step and the luminescence measurements will therefore be less sensitive than that of the first implementation. On the other hand, it is easier to implement since it comprises only a limited number of steps. Moreover, and as indicated above and in the experimental section, the inventors have verified that a moderate sonication does not disrupt the antigen-antibody bonds possibly present in the measuring medium (in particular when the donor and/or acceptor fluorescent compounds are conjugated to antibodies).

A third possibility, in the case where the process is carried out on isolated cells from a cell culture, is the sequence of the following steps:
a) distribution of the cells in the microplate,
b) addition of the fluorescent compounds (donor, acceptor, labeling agent) to the measuring medium,
c) reading of the microplate with a fluorimeter (donor signal, acceptor signal, labeling agent signal).

This implementation does not require sonication since the cells are uniformly distributed in the measuring medium, but requires the use of a labeling agent which is not rejected by living cells, such as Hoechst 33342.

A fourth possibility is the sequence of the following steps:
a) bringing the labeling agent into contact with cells or a tissue sample,
b) washing,
c) sonication,
d) transfer into a microplate (if the tissue or the cells were not already in a microplate),
e) addition of the fluorescent compounds (donor, acceptor) to the measuring medium,
f) reading of the microplate with a fluorimeter (donor signal, acceptor signal, labeling agent signal).
This protocol is preferred when working with adherent cells, for example in the context of tests intended to discover new medicaments. This protocol was used in Example 7.

### Reagent Kits

Finally, the invention relates to reagent kits suitable for carrying out the process according to the invention. The kits for carrying out the process according to which the luminescence of the labeling agent is measured at the same wavelength as that used to measure the luminescence of the long-life fluorescent compound comprise a set of reagents, including:
a long-life fluorescent compound;
a fluorescent labeling agent, the absorption spectrum of which allows the excitation thereof at the same wavelength as that used to excite the long-life fluorescent compound, and the emission spectrum of which allows the measurement of the luminescence thereof at the same wavelength as that used to measure the luminescence of the long-life fluorescent compound;
optionally, an acceptor fluorescent compound, the absorption spectrum of which is compatible with the emission spectrum of the long-life fluorescent compound, the long-life fluorescent compound and the acceptor fluorescent compound being FRET partners.

A preferred reagent kit is a kit in which:
the long-life fluorescent compound is chosen from: a europium cryptate; a europium chelate; a terbium chelate; a terbium cryptate;
the fluorescent labeling agent has an absorption spectrum which allows the excitation thereof of between 330 and 350 nm and an emission spectrum which allows the measurement of the luminescence thereof at the same wavelength as that used to measure the luminescence of the long-life fluorescent compound.

The kits for carrying out the process according to which the luminescence of the labeling agent is measured at the same wavelength as that used to measure the luminescence of the acceptor fluorescent compound comprise a set of reagents, including:
a long-life fluorescent compound;
an acceptor fluorescent compound, the absorption spectrum of which is compatible with the emission spectrum of the long-life fluorescent compound, the long-life fluorescent compound and the acceptor fluorescent compound being FRET partners;
a fluorescent labeling agent, the absorption spectrum of which allows the excitation thereof at the same wavelength as that used to excite the long-life fluorescent compound, and the emission spectrum of which allows the measurement of the luminescence thereof at the same wavelength as that used to measure the luminescence of the acceptor fluorescent compound.

Another preferred reagent kit is thus a kit in which:
the long-life fluorescent compound is chosen from: a europium cryptate; a europium chelate; a terbium chelate; a terbium cryptate;
the fluorescent labeling agent has an absorption spectrum which allows the excitation thereof between 330 and 350 nm and an emission spectrum which allows the measurement of the luminescence thereof at the same wavelength as that used to measure the luminescence of the acceptor fluorescent compound.

According to one implementation of the invention, the fluorescent labeling agent is chosen from the following compounds: a fluorescent intercalating agent, a fluorescent protein, a fluorescent compound which is a marker of mitochondria, a fluorescent compound which binds to the amine functions of proteins and a fluorescent compound which accumulates in lipid membranes. Examples of such labeling agents suitable for use according to the invention are: the Hoechst 33258 compound; the Hoechst 33342 compound; the Hoechst 34580 compound; DAPI (4′,6′-diamidino-2-phenylindole); green fluorescent protein (GFP), Mitotracker Orange, Mitotracker Green, rhodamine 123, dansyl chloride, nitrobenzoxadiazole and filipin.

The invention is illustrated by the following examples, given purely by way of indication.

### Example 1

### Obtaining of Tumor Tissue Cryosections

The method according to the invention was carried out on cryosections obtained from mouse tumors, induced by xenograft of NIH-3T3 cells overexpressing either the HER1 (EGFR) receptor or the HER2 (ErbB2) receptor, or both HER1 and HER2. These cells were obtained by transduction using a retroviral vector carrying the nucleic sequence encoding the proteins in question. This material constitutes a model close to a biopsy of human tumor tissue.

In order to obtain the retroviral construct carrying the HER1 gene, the EGFR sequence was extracted from the pCMV-XL4-EGFR vector (Origene-CliniSciences, Montrouge, France) by cleavage using the Not I restriction enzyme (combined with the Pvu I enzyme in order to cut the vector into fragments of size different than that of the sequence of interest) (Fermentas, Saint-Rémy-lès-Chevreuse, France). The sequence of interest was treated with T4 DNA polymerase (Roche Diagnostics, Meylan, France) in order to obtain blunt ends and to subsequently be inserted into the MSCV retroviral vector (Clontech-Ozyme) containing the puromycin resistance sequence (pMSCV-puro) digested beforehand with the Hpa I restriction enzyme and then dephosphorylated with CIAP “calf intestinal alkaline phosphatase” (Fermentas). Before being ligated with T4 DNA ligase (Roche), the sequences (insert and digested plasmid) were loaded onto a 1% agarose gel and, after electrophoresis, recovered according to their size and purified using the Nucleo Spin Extract II kit (Machery-Nagel, Hoerdt). Competent bacteria of E. coli DH5a type were subsequently transformed with the ligation product and then plated out on a Petri dish in LB agar medium. The colonies obtained were then placed in liquid LB medium in order to produce minipreparations of plasmids using the Nucleo Spin Plasmid Quick Pure kit (Machery-Nagel). The direction of insertion of the insert in the vector was verified by restriction analysis using the BgI II enzyme (Fermentas). In the same way, in order to obtain the retroviral construct carrying the HER2 gene, the HER2 sequence was extracted from the pCMV-XL4-HER2 vector (Origene-CliniSciences, Montrouge, France) and then inserted into the plasmid of the MSCV retroviral vector (Clontech-Ozyme) containing the hygromycin resistance sequence (pMSCV-hygro). In this case, the direction of insertion of the insert in the vector was verified by restriction analysis using the Xho I and BcI I enzymes (Fermentas). Finally, the pMSCV-puro-HER1 and pMSCV-hygro-HER2 plasmids were sent to the company Millegen (Labege, France) to be sequenced for verification of the absence of mutations in the HER1 or HER2 coding sequence. The pMSCV-puro-HER1 or pMSCV-hygro-HER2 vectors were transfected into an AmphoPack-293 packaging line (Clontech) for 24 h, allowing multiplication of the modified virus so as to produce capsulated viral particles. The supernatant containing the viral particles was collected, centrifuged and filtered through a 0.45 μm filter.

NIH/3T3 cells were cultured (2×10⁶ cells in a 150 mm dish) for 8 h, and then exposed for 16 h in the presence of a quarter of the volume of supernatant containing the viruses in DMEM medium containing 10% of fetal calf serum and polybrene (8 μg/ml, Sigma-Aldrich). After 24 h, the cells were washed and then selected by adding the antibiotic (400 μg/ml of hygromycin or 10 μg/ml of puromycin as appropriate) and incubating for two days. After selection and multiplication, the cells (5×10⁶) overexpressing either HER1 or HER2 or both HER1 and HER2 were administered by subcutaneous injection to athymic female mice, on their right flank. In what follows, the lines, and also the corresponding xenografts, expressing only HER1 will be called R1, the lines (and xenografts) expressing only HER2 will be called R2 and the lines (and xenografts) expressing both HER1 and HER2 will be called R1R2. The mice carrying tumors were sacrificed when the tumors reached a volume greater than 1500 mm³.

### Preparation of Xenograft Cryosections

After the animal was sacrificed, the tumor was excised, and the resulting surgical piece was immediately frozen by immersing it in liquid nitrogen, and then stored at −80° C.

In order to produce the cryosections, the metal sample-holder support of a cryotome was placed on dry ice for 1 min. A few drops of sterile water were subsequently deposited on the support, and then, without waiting, the surgical piece was deposited. By freezing, the water attached the sample to the support. Alternatively, the sample can be embedded in a gel of OCT (Optimal Cutting Temperature compound) type in place of the sterile water. The frozen sample was then cut at the desired thickness with the microtome contained in the cryostat (−25° C.) and the still-frozen sections (cryosections) were collected in eppendorf tubes precooled in dry ice. The thickness of the cryosections was generally between 10 μm and 50 μm. For the applications below, a thickness of 50 μm was considered to be optimal in terms of handling practicality.

### Example 2

### Measurement of the Emission Signal of an Intercalating Agent Allowing the Measurement of the Amount of Cells in a Section of a Tumor Cell Tissue

A cryosection from a mouse tumor having undergone a xenograft of R1R2 cells, prepared according to the procedure described in Example 1, and called cryosection R1R2, was placed in an eppendorf tube in 180 μl of PBS buffer, to which 20 μl of a solution of Hoechst 33342 (Invitrogen # H1399 in solution 2 mg/ml DMSO) prediluted to 1/100 in PBS buffer, pH 7 (i.e. a concentration of Hoechst 33342 of 3.25 μM), were added. After incubation for 10 min in the dark, centrifugation and a washing with 3 times 400 μl of PBS buffer+0.05% Tween® 20 were carried out. Finally, the pellet formed by the cryosection was taken up in 300 μl of PBS buffer containing 0.1% of BSA (bovine serum albumin) and a protease inhibitor (“complete mini” Roche #04 693 124 001; 1 tablet/10 ml). The tube was kept in ice and subjected to sonication (5 s at 20% of the power with a Branson 450 sonicator provided with a “micro tip” probe). 5 μl, 20 μl and 100 μl of the homogeneous suspension obtained were then deposited in wells of a microplate (Costar #3694 “96 well half area flat bottom black polystyrene”), and made up to 100 μl with the same buffer as that used for the sonication step described later.

The microplate was then placed in a Tecan Saphir2 fluorimeter. After excitation of the wells at 340 nm, the fluorescence of the Hoechst 33342 intercalating agent was measured at the three wavelengths of 490 nm, 545 nm and 590 nm (H490, H545 and H590), corresponding to the main emission peaks of terbium, the 490 nm wavelength also corresponding to one of the emission peaks of europium. The fluorescence was in this case measured in continuous fluorescence mode, i.e. immediately after excitation, and for the shortest time permitted by the instrument (20 μs). The results are given in Table 2.

TABLE 2

| R1R2 cells (μl) | 5 | 20 | 100 |
|---|---|---|---|
| H490 | 2610 | 10 770 | 52 080 |
| H545 | 3240 | 11 940 | 48 080 |
| H590 | 3260 | 11 670 | 43 980 |

These results show that the fluorescence intensity as a function of the cell suspension volumes changes in a linear manner, and is therefore proportional to the amount of cells present in the wells. This example demonstrates that the fluorescence emitted by the Hoechst 33342—DNA complex can be measured at 490, 545 or 590 nm, in addition to being at the wavelengths conventionally used (excitation at 330-380 nm and measurement of emission at 430-530 nm).

### Example 3

### Normalization of the Terbium Emission Signal Allowing the Measurement of the Expression of a Receptor in a Section of a Tumor Cell Tissue

### 3.1. Labeling of an Anti-HER2 Antibody with a Terbium Cryptate

The FRP5 antibody (supplied by the Institut de Recherche en Cancérologie [Cancerology Research Institute] of Montpellier, IRCM) (150 μl at 1.2 mg/ml) was desalified on a NAPS (G25) column equilibrated in a 50 mM phosphate buffer, pH 8. The excluded fraction was collected in the form of an eluate of 400 μl at 0.45 mg/ml (i.e. 1.2 nmol) in phosphate buffer, pH 8. 12 nmol (i.e. 10 eq.) of Lumi4Tb-NHS (terbium cryptate, Cisbio Bioassays) in solution in 1.4 μl of DMSO were added to this fraction. After incubation for 1 h at 20° C., the labeled antibody was purified on NAPS (G25) column equilibrated in 100 mM phosphate buffer, pH 7. The excluded fraction (400 μl) is collected and the FRP5 antibody labeled at a rate of 5.3 cryptates per antibody is thus obtained. The solution was frozen in the presence of 0.1% of BSA until its use.

### 3.2. Measurement of the Expression of HER2 (ErbB2) by Terbium Emission Time-Resolved Fluorescence Measurement at 490 nm and Normalization by Fluorescence of an Intercalating Agent, Also Measured at 490 nm in Continuous Fluorescence Mode

An R1R2 cryosection prepared in Example 1 was placed in an eppendorf tube in 180 μl of PBS buffer, pH 7, containing 4% of BSA (protease-free quality) and a protease inhibitor (Roche, 1 tablet/10 ml). The FRP5-Lumi4Tb antibody prepared in Example 3.1 was added so as to obtain a final antibody concentration of 50 nM in the eppendorf tube. After incubation overnight at 30° C., 20 μl of a solution of Hoechst 33342 (Invitrogen # H1399 in solution 2 mg/ml DMSO) prediluted to 1/100 in buffer, pH 7, were added. After incubation for 10 min in the dark, the tube was centrifuged and washed with 3 times 400 μl of PBS buffer+0.05% Tween® 20. The pellet formed by the cryosection was taken up in 300 μl of PBS buffer containing 0.1% of BSA and a protease inhibitor (“complete mini” Roche #04 693 124 001; 1 tablet/10 ml). The tube was kept in ice and subjected to sonication (5 s at 20% of the power with a Branson 450 sonicator provided with a “micro tip” probe). 5 μl, 20 μl and 100 μl of the homogeneous suspension obtained were deposited in wells of a microplate (Costar #3694 “96 well half area flat bottom black polystyrene”) and made up to 100 μl with the same buffer as that used for the sonication step.

The plate was then placed on a Tecan Saphir2 instrument provided with excitation via a flashlamp and with a 337 nm excitation filter.

The continuous mode fluorescence, corresponding to that of Hoechst 33342, was measured at 490 nm, without delay after excitation, and for an integration time of 20 μs. The time-resolved fluorescence (TRF), corresponding to the signal emitted by the terbium cryptate, was also measured at 490 nm but after a delay of 100 μs following excitation, and for an integration time of 400 μs.

After subtraction of the background noise measured on buffer alone, the values given in Table 3 are obtained, in which table:

TRF490 represents the time-resolved measurement of the terbium cryptate signal at 490 nm,
H490 represents the continuous-fluorescence measurement of the Hoechst 33342 signal at 490 nm, and
normalized TRF490 represents the ratio of TRF 490 to H490 modified by an appropriate multiplying coefficient (in this example ×1000) so as to have a whole number of the same order of magnitude as the crude fluorescence levels measured.

TABLE 3

| R1R2 cells (μl) | 5 | 20 | 100 |
|---|---|---|---|
| TRF490 | 2141 | 9158 | 38 497 |
| H490 | 1284 | 6753 | 27 259 |
| Normalized TRF490 | 16 670 | 13 560 | 14 120 |

It is observed that the TRF490 value increases proportionally to the volume of cell suspension added to the wells, whereas the normalized TRF 490 value is constant overall—on average 14 800 afu (arbitrary fluorescence units)—and represents a measurement of the expression level of the HER2 receptor expressed by the R1R2 cells.

The same experiment carried out on sections obtained from the R1 cell line overexpressing HER1 alone (negative control) and not overexpressing HER2 makes it possible to estimate the specificity and the background noise of the detection of this overexpression by the technique described above. After depositing in wells on the same plate and reading under the same conditions, the following values, reported in Table 4, are observed.

TABLE 4

| R1 cells (μl) | 5 | 20 | 100 |
|---|---|---|---|
| TRF490 | 20 | 189 | 289 |
| H490 | 1284 | 6753 | 27 259 |
| Normalized TRF490 | 156 | 280 | 106 |

A normalized TRF 490 value, the average of which is 180 afu for this negative control, which represents a signal/noise ratio of about 80, is observed in this case.

This example demonstrates that the method according to the invention makes it possible to normalize the signal emitted by a terbium cryptate with respect to the amount of cells present in the medium, by measuring the fluorescence emitted by this cryptate and the Hoechst 33342 at the same wavelength (490 nm), but in different time windows.

### 3.3. Measurement of the Expression of HER2 (ErbB2) by Terbium Emission Time-Resolved Fluorescence Measurement at 545 nm and Normalization by Fluorescence of an Intercalating Agent, Measured at 545 nm in Continuous Fluorescence Mode

An R1R2 cryosection prepared in Example 1 was treated as in Example 3.2.

The signal was read in continuous fluorescence mode at 545 nm and in TRF mode at 545 nm on a Tecan Saphir2 instrument provided with excitation by flashlamp, the monochromator of which was set at 340 nm (bandwidth 20 nm) for excitation and at 545 nm (bandwidth 10 nm) for emission. The time-resolved measurement was carried out after a delay of 100 μs following excitation and for an integration time of 500 μs; the continuous fluorescence measurement was carried out without delay and for the minimum integration time possible on the instrument (20 μs).

After subtraction of the background noise measured on buffer alone, the values given in Table 5 are obtained, in which table:

TRF545 represents the time-resolved measurement of the terbium signal at 545 nm,
H545 represents the continuous fluorescence measurement of the Hoechst 33342 signal at 545 nm, and
normalized TRF545 represents the ratio of TRF 545 to H545 modified by an appropriate multiplying coefficient (in this example ×10000) so as to have a whole number of the same order of magnitude as the crude fluorescence levels measured.

TABLE 5

| R1R2 cells (μl) | 5 | 20 | 100 |
|---|---|---|---|
| TRF545 | 298 | 1227 | 4320 |
| H545 | 3120 | 9470 | 28 840 |
| Normalized TRF545 | 954 | 1295 | 1499 |

It is observed that the H545 value is proportional to the volume of cell suspension added. It is also observed that the TRF545 value increases proportionally to the volume of cell suspension added to the wells, whereas the normalized TRF 545 value is constant overall, on average 1250 afu, and represents a measurement of the expression level of the HER2 receptor expressed by the R1R2 cells.

This example demonstrates that the method according to the invention makes it possible to normalize the signal emitted by a terbium cryptate with respect to the amount of cells present in the medium, by measuring the fluorescence emitted by this cryptate and the Hoechst 33342 at the same wavelength (545 nm), but in different time windows.

### 3.4. Measurement of the Expression of HER2 (ErbB2) by Terbium Emission Time-Resolved Fluorescence Measurement at 590 nm and Normalization by Fluorescence of an Intercalating Agent, Measured at 590 nm in Continuous Fluorescence Mode

Example 3.3 was reproduced, but this time the luminescence of the terbium cryptate and of the Hoechst 33342 was measured at 590 nm. The values obtained are given in Table 6, in which:

TRF590 represents the time-resolved measurement of the terbium signal at 590 nm,
H590 represents the continuous fluorescence measurement of the Hoechst 33342 signal at 590 nm, and
normalized TRF590 represents the ratio of TRF 590 to H590 modified by an appropriate multiplying coefficient (in this example ×1000) so as to have a whole number of the same order of magnitude as the crude fluorescence levels measured.

TABLE 6

| R1R2 cells (μl) | 5 | 20 | 100 |
|---|---|---|---|
| TRF590 | 434 | 1159 | 3851 |
| H590 | 2854 | 8491 | 25 992 |
| Normalized TRF590 | 1521 | 1365 | 1481 |

Here again, the TRF590 value increases proportionally to the volume of cell suspension added to the wells, whereas the normalized TRF 590 value is constant overall, on average 1450 afu, and represents a measurement of the expression level of the HER2 receptor expressed by the R1R2 cells.

This example demonstrates that the method according to the invention makes it possible to normalize the signal emitted by a terbium cryptate with respect to the amount of cells present in the medium, by measuring the fluorescence emitted by this cryptate and the Hoechst 33342 at the same wavelength (590 nm), but in different time windows.

### Example 4

### Normalization of the Europium Emission Signal Allowing the Measurement of the Expression of a Receptor in a Section of a Tumor Cell Tissue

### 4.1. Labeling of an Anti-HER2 Antibody with Europium Cryptate

10 μl of a 0.1 M NaHPO₄ solution (pH 9) and then 40 μl of a 0.1 M PO₄ solution (pH 8), were added to an FRP5 antibody (IRCM/Montpellier) (150 μl at 1.2 mg/ml in PBS, pH 7), so as to obtain a solution containing 1.3 nmol of FRP5 in 200 μl, i.e. a final concentration of 6.5 μM in a pH 8 buffer. 13.5 nmol of EuTBP-NHS (i.e. 10 eq. of europium cryptate, Cisbio Bioassays) were added to this solution. After incubation for 1 h at 20° C., this solution was purified on a NAPS (G25) column equilibrated in 100 mM phosphate buffer, pH 7. The excluded fraction (300 μl) was collected so as to obtain a solution containing the FRP5-TBPEu antibody labeled at a rate of 3.4 cryptates per antibody. This solution was frozen in the presence of 0.1% of BSA until its use.

### 4.2. Measurement of the Expression of HER2 by Europium Emission Time-Resolved Fluorescence Measurement at 585 nm and Normalization by Fluorescence of an Intercalating Agent, Also Measured at 585 nm in Continuous Fluorescence Mode

Example 3.2 was reproduced with the antibody prepared in Example 4.1. The fluorescence is measured at the wavelength of 585 nm using a Rubystar fluorimeter (BMG), provided with excitation at 337 nm by a nitrogen laser.

The time-resolved measurement, corresponding to the europium cryptate signal, was carried out after a delay of 100 μs following excitation and for an integration time of 400 μs. The continuous fluorescence measurement of the Hoechst 33342 signal is carried out without delay after excitation and for the minimum integration time possible on the instrument (10 μs).

After subtraction of the background noise measured on buffer alone, the values given in Table 7 are obtained, in which table:

TRF585 represents the time-resolved measurement of the europium cryptate signal at 585 nm,
H585 represents the continuous fluorescence measurement of the Hoechst 33342 signal at 585 nm, and
normalized TRF585 represents the ratio of TRF 585 to H585 modified by an appropriate multiplying coefficient (in this example×200) so as to have a whole number of the same order of magnitude as the crude fluorescence levels measured.

TABLE 7

| R1R2 cells (μl) | 5 | 20 | 100 |
|---|---|---|---|
| TRF585 | 13 | 21 | 64 |
| H585 | 186 | 404 | 824 |
| Normalized TRF585 | 14 | 10 | 16 |

It is observed that the TRF585 value increases proportionally to the volume of cell suspension added to the wells, whereas the normalized TRF 585 value is constant overall, on average 13 afu, and represents measurement of the expression level of the HER2 receptor expressed by the R1R2 cells.

This example demonstrates that the method according to the invention makes it possible to normalize the signal emitted by a europium cryptate with respect to the amount of cells present in the medium, by measuring the fluorescence emitted by this cryptate and the Hoechst 33342 at the same wavelength (585 nm), but in different time windows.

### Example 5

### Normalization of the Terbium Emission Signal Allowing the Measurement of the Expression of a Receptor by TR-FRET in a Section of a Tumor Cell Tissue

### 5.1. Labeling of an Anti-HER1 Antibody with a Terbium Cryptate

An anti-HER1 antibody REGF01 (Cisbio Bioassays) (92 μl at 5.4 mg/ml) was desalified on a NAPS (G25) column equilibrated in a 50 mM phosphate buffer, pH 8. The excluded fraction was collected in the form of an eluate of 400 μl at 1 mg/ml (i.e. 3 nmol) in phosphate buffer, pH 8. 20 nmol (i.e. 7 eq.) of Lumi4Tb-NHS (terbium cryptate, Cisbio Bioassays) in solution in 2.3 μl of DMSO were added to this fraction. After incubation for 1 h at 20° C., the labeled antibody was purified on NAPS (G25) column equilibrated in 100 mM phosphate buffer, pH 7. The excluded fraction (400 μl) was collected so as to thus obtain the REGF01 antibody labeled at a rate of 4.4 cryptates per antibody. The solution was frozen in the presence of 0.1% of BSA until its use.

### 5.2. Labeling of an Anti-HER1 Antibody with a FRET Acceptor

An anti-HER1 antibody REGF08 (Cisbio Bioassays) (200 μl at 1.6 mg/ml) was dialyzed against a 50 mM phosphate buffer, pH 8 (overnight at 4° C.). After dialysis, the fraction collected in phosphate buffer, pH 8, contains the antibody at 0.93 mg/ml. 4 nmol (i.e. 5 eq.) of activated acceptor fluorophore (d2-NHS; Cisbio Bioassays) in solution in 3 μl of DMSO were added to 130 μl of this fraction (i.e. 0.8 nmol). After incubation for 1 h at 20° C., this solution was purified on a NAPS (G25) column equilibrated in 100 mM phosphate buffer, pH 7. The excluded fraction (400 μl) was collected so as to thus obtain the REGF08 antibody labeled at a rate of 3.3 fluorophores per antibody. The solution was frozen in the presence of 0.1% of BSA until its use.

### 5.3. Measurement of the Expression of HER1 (EGFR) Receptors by Measurement of TR-FRET of the Emission of Terbium at 490 nm and of the Acceptor at 665 nm and Normalization by Fluorescence of an Intercalating Agent, Also Measured at 490 nm in Continuous Fluorescence Mode

### 5.3.1

An R1R2 cryosection prepared in Example 1 was placed in an eppendorf tube in 180 μl of PBS buffer, pH 7, containing 4% of BSA (protease-free quality) and a protease inhibitor (Roche, 1 tablet/10 ml). The REGF01-Lumi4Tb and REGF08-d2 antibodies prepared in Examples 5.1 and 5.2 were added so as to obtain a final concentration of 50 nM of each antibody in the tube. After incubation overnight at 30° C., 20 μl of a solution of Hoechst 33342 (Invitrogen # H1399 in solution 2 mg/ml DMSO) prediluted to 1/100 in buffer, pH 7, were added. After incubation for 10 min in the dark, the tube was centrifuged and washed with 3 times 400 μl of PBS buffer+0.05% Tween® 20. The pellet formed by the cryosection was taken up in 300 μl of PBS buffer containing 0.1% of BSA and a protease inhibitor (“complete mini” Roche #04 693 124 001; 1 tablet/10 ml). The tube was kept in ice and subjected to sonication (5 s at 20% of the power with a Branson 450 sonicator provided with a “micro tip” probe). 10 μl, 20 μl, 40 μl and 100 μl of the homogeneous suspension obtained were deposited in wells of a microplate (Costar #3694 “96 well half area flat bottom black polystyrene”) and made up to 100 μl with the same buffer as that used for the sonication step.

The plate was then placed on a Tecan Saphir2 instrument provided with excitation by a flashlamp and with a monochromator set at 340 nm (bandwidth 20 nm) for excitation. For the time-resolved (TRF) measurements, the fluorescence was measured at 490 nm after a delay of 100 μs following excitation, and for an integration time of 400 μs; for the continuous fluorescence measurement of the Hoechst 33342 fluorescence, the fluorescence was measured at 490 nm without delay after excitation, and for the minimum integration time possible on the instrument (20 μs).

After subtraction of the background noise measured on buffer alone, the values given in Table 8 are obtained, in which table:

TRF490 represents the time-resolved measurement of the terbium signal at 490 nm,
H490 represents the continuous fluorescence measurement of the Hoechst 33342 signal at 490 nm, and
normalized TRF490 represents the ratio of TRF 490 to H490 modified by an appropriate multiplying coefficient (in this example ×1000) so as to have a whole number of the same order of magnitude as the crude fluorescence levels measured.

TABLE 8

| R1R2 cells (μl) | 10 | 20 | 40 | 100 |
|---|---|---|---|---|
| TRF490 | 3850 | 10 430 | 19 340 | 59 740 |
| H490 | 3420 | 9770 | 18 040 | 49 480 |
| Normalized | 11 260 | 10 670 | 10 720 | 12 070 |
| RF490 | | | | |

It is observed that the TRF490 value increases proportionally to the volume of cell suspension added to the wells, whereas the normalized TRF 490 value is constant overall, on average 11 180 afu, and represents a measurement of the expression level of the HER1 receptor expressed by the R1R2 cells.

### 5.3.2. Evaluation of the Contribution of Terbium Cryptate to the Signal Measured at 665 nm: Determination of the “Reagent Blank” 8665

To measure the expression of the HER1 receptor by TR-FRET, increase in emission of the acceptor at 665 nm can be measured. Nevertheless, the fluorescence measured at 665 nm does not correspond only to the luminescence of the acceptor, since terbium cryptate is also weakly fluorescent at this wavelength. Thus, it is necessary to correct the signal measured at 665 nm. For this, various concentrations of REGF08-d2 antibody and of REGF0-Lumi4Tb antibody (0.2 nM, 1 nM, 5 nM) are brought together, in the absence of Hoechst 33342 and of cells (mixture hereinafter referred to as “reagent blank”), and time-resolved measurement of the signals emitted at 490 and at 665 nm is carried out. The results given in Table 9 are obtained, in which table:

TRF490 represents the signal measured in time-resolved mode at 490 nm, corresponding essentially to the emission of terbium cryptate, and
B665 represents the signal measured in time-resolved mode at 665 nm, corresponding essentially to the residual emission of terbium cryptate at this wavelength.

TABLE 9

| | Antibody concentration (nM) | | |
|---|---|---|---|
| | 0.2 | 1 | 5 |
| TRF490 | 1604 | 8387 | 42 959 |
| B665 | 131 | 193 | 769 |

These results make it possible to determine the relationship between the luminescence of terbium cryptate at 490 nm and that at 665 nm. This relationship is a linear function which makes it possible to calculate, in the experiments hereinafter, and using the TRF490 value measured on the sample, that of B665 in this sample.

### 5.3.3

Example 5.3.1 is reproduced, but this time by measuring in addition the signal in time-resolved mode at 665 nm (TRF665), corresponding essentially to the emission of the acceptor compound d2 following the transfer of energy between the anti-HER1-Lumi4Tb antibody and the anti-HER1-d2 antibody.

The results are given in Table 10.

TABLE 10

| R1R2 cells (μl) | 10 | 20 | 40 | 100 |
|---|---|---|---|---|
| TRF665 | 1260 | 3840 | 7430 | 25 060 |
| TRF490 | 6850 | 10 430 | 19 340 | 59 740 |
| B665 | 146 | 250 | 390 | 1030 |
| Delta F665 = TRF665- | 1113 | 3594 | 7037 | 24 028 |
| B665 | | | | |
| H490 | 3420 | 9770 | 18 040 | 49 480 |
| Normalized Delta F665 = | 3260 | 3680 | 3900 | 4860 |
| Delta F665/H490 | | | | |

It is observed that the Delta F 665 value increases proportionally to the volume of cell suspension added to the wells, whereas the normalized Delta F 665 value is constant overall, on average 3920 afu, and represents a measurement of the expression level of the HER1 receptor expressed by the R1R2 cells and measured by means of the TR-FRET process.

It will be noted that this measurement of the expression using two antibodies is less subject to parasitic phenomena such as possible nonspecific interactions between antibodies and target proteins, the measurement being in this case based on a double specificity provided by the use of two antibodies.

### 5.3.4

The same plate was placed on a filter-based instrument of EnVision type provided with excitation via a flashlamp and with a monochromator set at 340 nm (bandwidth 20 nm) for excitation. For the time-resolved (TRF) measurements, the fluorescence was measured (i) at 490 nm after a delay of 100 μs following excitation, and for an integration time of 400 μs; and (ii) at 665 nm after a delay of 60 μs following excitation, and for an integration time of 400 μs. For the continuous fluorescence measurement of Hoechst 33342 fluorescence, the fluorescence was measured at 490 nm without delay after excitation, and for the minimum integration time possible on the instrument (20 μs).

The results are given in Table 11.

TABLE 11

| | R1R2 cells (μl) | | | |
|---|---|---|---|---|
| | 10 | 20 | 40 | 100 |
| H490 | 250 000 | 683 900 | 1 530 000 | 3 634 000 |
| TRF490 | 116 000 | 250 900 | 454 300 | 1 211 000 |
| Normalized TRF490 | 46 390 | 36 690 | 29 690 | 33 330 |
| TRF665 | 10 900 | 25 090 | 50 400 | 152 800 |
| B665 | 650 | 1400 | 2540 | 6780 |
| Delta F665 | 10 260 | 23 690 | 47 880 | 146 000 |
| Normalized Delta | 4100 | 3460 | 3130 | 4020 |
| F665 | | | | |

These results are similar to those obtained with the Tecan Saphir2 fluorimeter in Example 5.3.3, even though the limited sensitivity can be very different between a monochromator instrument such as the Tecan Saphir2 and a filter-based instrument such as EnVision.

### 5.5. Measurement of the Expression of HER1 (EGFR) Receptors by Measurement of TR-FRET of the Emission of Terbium at 545 nm and of the Acceptor at 665 nm and Normalization by Fluorescence of an Intercalating Agent, Also Measured at 545 nm in Continuous Fluorescence Mode

Example 5.3.2 was reproduced, but this time the luminescence of the terbium cryptate and of the Hoechst 33342 was measured on a Tecan Saphir2 instrument provided with excitation via a flashlamp, the monochromator of which was set at 340 nm (bandwidth 20 nm) for excitation, with the monochromator at 545 nm (bandwidth 10 nm) for emission. The time-resolved fluorescence was measured after a delay of 100 μs following excitation and for an integration time of 500 μs, and the continuous fluorescence measurement of Hoechst 33342 fluorescence was carried out without delay after excitation and for the minimum integration time possible on the instrument (20 μs).

The residual emission of the terbium cryptate at 665 nm in the absence of FRET (B665) was determined as in Example 5.3.2. The results obtained are given in Table 12, in which: TRF545 represents the time-resolved measurement of the terbium signal at 545 nm, and H545 represents the continuous fluorescence measurement of the Hoechst 33342 signal at 545 nm.

TABLE 12

R1R2 cells (μl)

| | 10 | 20 | 40 | 100 |
|---|---|---|---|---|
| TRF545 | 3250 | 9330 | 16 350 | 51 240 |
| B665 | 227 | 355 | 500 | 1 240 |
| TRF665 | 1380 | 4180 | 7100 | 24 780 |
| Delta F665 = | 1150 | 3820 | 6600 | 23 540 |
| TRF665 − B665 | | | | |
| H545 | 3420 | 9920 | 18 500 | 51 150 |
| Normalized Delta | 3370 | 3850 | 3570 | 4600 |
| F665 | | | | |

Results similar to those previously obtained are observed, namely an increase in the TRF665 value proportionally to the volume of cell suspension added to the wells, whereas the normalized TRF 665 value is constant overall, on average 3850 afu, and represents a measurement of the expression level of the HER1 receptor expressed by the R1R2 cells.

This example shows that the invention can be carried out by detecting the signals of the terbium cryptate and of the intercalating agent at 545 nm.

### 5.6. Measurement of the Expression of HER1 (EGFR) Receptors by Measurement of TR-FRET of the Emission of Terbium at 590 nm and of the Acceptor at 665 nm and Normalization by Fluorescence of an Intercalating Agent, Measured at 590 nm in Continuous Fluorescence Mode

Example 5.5 was reproduced, but this time the luminescence of the terbium cryptate and that of the Hoechst 33342 product were measured at 590 nm. The results obtained are given in Table 13, in which:

TRF590 represents the time-resolved measurement of the terbium signal at 590 nm, and
H590 represents the continuous fluorescence measurement of the Hoechst 33342 signal at 590 nm.

TABLE 13

R1R2 cells (μl)

| | 10 | 20 | 40 | 100 |
|---|---|---|---|---|
| TRF590 | 2840 | 7420 | 13 215 | 39 940 |
| B665 | 71 | 178 | 313 | 935 |
| TRF665 | 1100 | 3370 | 6400 | 21 900 |
| Delta F665 | 1030 | 3190 | 6090 | 20 970 |
| H590 | 3410 | 9930 | 18 090 | 51 020 |
| Delta F665/H590 | 3020 | 3210 | 3365 | 4110 |

These results are similar to those previously obtained and show that the invention can be carried out by measuring the luminescence of the terbium cryptate and of the Hoechst 33342 at 590 nm.

### Example 6

### Normalization of the TR-FRET Emission Signal Allowing the Measurement of the Dimerization of a Receptor in a Section of a Tumor Cell Tissue

### 6.1. Labeling of an Anti-HER2 Antibody with a Terbium Cryptate

The anti-HER2 antibody trastuzumab (trade name “Herceptin”) was labeled with the terbium cryptate Lumi4Tb, as described in Example 5.1.

### 6.2. Labeling of an Anti-HER1 Antibody with a FRET Acceptor

The anti-HER1 antibody cetuximab (trade name “Erbitux”) was labeled with a FRET acceptor, “d2”, as described in Example 5.2.

### 6.3. Measurement of the Heterodimerization of HER1-HER2 Receptors by Measurement of TR-FRET of the Emission of Terbium at 545 nm and of the Acceptor at 665 nm and Normalization by the Fluorescence of an Intercalating Agent, Measured at 545 nm in Continuous Fluorescence Mode

In this example, the signal emitted at 665 nm results from a transfer of energy between the two labeled antibodies, when they bind to an HER1-HER2 dimer.

Two R1, or else R2, or else R1R2, cryosections prepared in Example 1 and 50 μm thick were placed in an eppendorf tube in 180 μl of PBS buffer, pH 7, containing 10% of BSA (protease-free quality) and a protease inhibitor (Roche, 1 tablet/10 ml). The trastuzumab-Lumi4Tb and cetuximab-d2 antibodies prepared in Examples 6.1 and 6.2 were added so as to obtain a final concentration of 50 nM of each antibody. After incubation overnight at 37° C., 20 μl of a solution of Hoechst 33342 (Invitrogen # H1399 in solution 2 mg/ml DMSO) prediluted to 1/100 in buffer, pH 7, were added. After incubation for 5 min in the dark, the tube was centrifuged for 2 min at 12 000 rpm and washed with 3 times 400 μl of PBS buffer+0.1% of BSA.

The pellet formed by the cryosection was taken up in 300 μl of PBS buffer containing 0.1% of BSA and a protease inhibitor (“complete mini” Roche #04 693 124 001; 1 tablet/10 ml). The tube was kept in ice and subjected to sonication (5 s at 20% of the power with a Branson 450 sonicator provided with a “micro tip” probe). 100 μl of the homogeneous suspension obtained were deposited (in duplicate) in wells of a microplate (Costar #3694 “96 well half area flat bottom black polystyrene”).

The plate was then placed on a Tecan Infinite F500 instrument provided with excitation via a flashlamp and with 340 nm filter (bandwidth 20 nm) for excitation. The continuous fluorescence reading is carried out with a 544 nm filter (bandwidth 25 nm) and the TRF reading is carried out at 544 nm (bandwidth 25 nm) (given the band width, equivalent to a measurement at 545 nm) and 665 nm (bandwidth 5 nm).

The time-resolved measurement was carried out after a delay of 100 μs following excitation and for an integration time of 400 μs, and the continuous fluorescence measurement of Hoechst 33342 fluorescence was carried out without delay after excitation and for the minimum integration time possible on the instrument (20 μs).

The contribution of the terbium cryptate at 665 nm was calculated for each measurement as in Example 5.3.2. The results obtained after subtraction of the background noise measured on buffer alone are given in Table 14.

TABLE 14

| | Samples (100 μl) | | |
|---|---|---|---|
| | R1R2 | R2 | R1 |
| Hoechst | 9870 | 37 420 | 11 300 |
| TRF545 | 4950 | 16 450 | 2350 |
| Normalized TRF545 | 5010 | 4400 | 2080 |
| B665 | 4660 | 15 500 | 2220 |
| TRF665 | 42 400 | 13 180 | 2100 |
| Delta F665 = TRF665 − B665 | 37 730 | −2320 | −116 |
| Normalized Delta F665 | 38 200 | −621 | −102 |

The TRF 545 signal is representative of the binding of the anti-HER2 antibody (trastuzumab-Lumi4Tb), and therefore of the expression of the R2 receptor. The normalization of this signal by the method according to the invention makes it possible to compare the HER2 expression levels in various samples: thus, the normalized TRF545 signal appears to be equivalent in the R1R2 and R2 samples, whereas it was 3 times higher in the R2 sample than in the R1R2 sample before normalization (TRF545).

The normalized Delta F665 signal is representative of the presence of heterodimers between HER1 and HER2 receptors: the normalized Delta F665 values confirm the presence of heterodimers in the R1R2 sample and their absence in the R1 and R2 samples.

### 6.4. Measurement of the Heterodimerization of HER1-HER2 Receptors by Measurement of TR-FRET of the Emission of Terbium at 490 nm and of the Acceptor at 665 nm and Normalization by the Fluorescence of an Intercalating Agent, Measured at 490 nm in Continuous Fluorescence Mode

The same plate was placed on a Pherastar FS filter-based instrument provided with excitation via a flashlamp and with filters available in the form of optical modules; in order to obtain the fluorescence values at 665 nm, it is necessary to carry out an independent measurement by changing the optical module. For the time-resolved (TRF) measurements, the fluorescence was measured (i) at 490 nm after a delay of 100 μs following excitation, and for an integration time of 400 μs; and (ii) at 665 nm after a delay of 100 μs following excitation, and for an integration time of 400 μs. For the continuous fluorescence measurement of Hoechst 33342 fluorescence, the fluorescence was measured at 490 nm without delay after excitation, and for the minimum integration time possible on the instrument (20 μs).

The results obtained are given in Table 15.

TABLE 15

| | Samples (100 μl) | | |
|---|---|---|---|
| | R1R2 | R2 | R1 |
| H490 | 4660 | 21 900 | 6270 |
| TRF490 | 142 880 | 560 250 | 64 150 |
| Normalized TRF490 | 306 609 | 255 822 | 102 313 |
| B665 | 3942 | 15 419 | 1777 |
| TRF665 | 27 387 | 7496 | 1126 |
| Delta F665 = TRF665 − | 23 445 | −7923 | −651 |
| B665 | | | |
| Normalized Delta F665 | 50 312 | −3618 | −1038 |

These results are similar to those of Example 6.3.

### Example 7

### TR-FRET Immunoassay Subsequent to Normalizing Labeling

In this example, the protocol used varies from that of the previous examples: it comprises the following steps: bringing the intercalating agent into contact with cells or a tissue sample, washing, sonication, transfer into a microplate, addition of the fluorescent compounds (donor, acceptor) to the measuring medium, reading of the microplate with a fluorimeter (donor signal, acceptor signal, intercalating agent signal).

An R1R2 cryosection 50 μm thick, prepared in Example 1, was placed in an eppendorf tube in 270 μl of PBS buffer, pH 7, containing 0.1% of BSA (protease-free quality) and a protease inhibitor (Roche, 1 tablet/10 ml). 30 μl of a solution of Hoechst 33342 (Invitrogen # H1399 in solution 2 mg/ml DMSO) prediluted to 1/100 in buffer, pH 7, were then added. After incubation for 10 min in the dark, the tube was centrifuged for 2 min at 12 000 rpm and washed with 3 times 400 μl of PBS buffer+0.1% of BSA.

The pellet formed by the cryosection was taken up in 300 μl of PBS buffer containing 0.1% of BSA and a protease inhibitor (“complete mini” Roche #04 693 124 001; 1 tablet/10 ml). The tube was kept in ice and subjected to sonication (5 s at 20% of the power with a Branson 450 sonicator provided with a “micro tip” probe) and the homogenate obtained was diluted three times. 20 μl, 40 μl and 80 μl of this suspension were deposited (in duplicate) in wells of a microplate (Costar #3694 “96 well half area flat bottom black polystyrene”) and made up with buffer so as to obtain a total volume of 80 μl per well (respectively referred to as H20, H40 and H80). Wells B20, B40 and B80 were prepared according to the same procedure but with the Hoechst 33342 being omitted, so as to constitute a “blank” signal.

A solution of a mixture of two labeled antibodies, cetuximab-Lumi4Tb (anti-HER1 antibody labeled at a rate of 7.5 Lumi4Tb terbium cryptates per antibody according to the protocol described in Example 5.1) and the Ab10-d2 antibody (ThermoFisher anti-HER1 antibody Ab10 labeled at a rate of 2.1 “d2” FRET acceptors per antibody according to a protocol described in Example 5.2), was added to all the wells so as to obtain a final concentration in the wells of 1 nM of cetuximab-Lumi4Tb and 2 nM of Ab10-d2.

The fluorescence of the Hoechst 33342 compound was then measured with a 490 nm filter (10 nm) for emission and a 337 nm filter (20 nm) for excitation on a Pherastar FS instrument equipped with a flashlamp, using the “advanced TRF” mode and slicing into delays of 5 μs.

After exportation of the data into software of “spreadsheet” type, the data are reprocessed by calculating the fluorescence intensities measured at 490 nm corresponding to the 30-45 μs time windows (H490 signal) and adding up the numbers obtained in each elementary interval of 5 μs.

These measurements in fact showed that, on this particular instrument, it is necessary to measure the Hoechst 33342 fluorescence on the 30-45 μs time window, and to avoid measuring the immediate signal (0-30 μs) which causes a saturation of the reader. It would also have been possible to directly measure the Hoechst 33342 fluorescence with a delay of 30 μs and an integration time of 15 μs.

The time-resolved fluorescence at 490 nm (TRF490, corresponding essentially to the terbium cryptate signal) and that at 665 nm (TRF665, fluorescence of the acceptor) were measured as in Example 6.3, but with a delay of 60 μs and an integration time of 340 μs.

The results obtained are given in Table 16.

TABLE 16

| | R1R2 cells (μl) | | |
|---|---|---|---|
| | 20 | 40 | 80 |
| TRF 490 | 212 682 | 174 278 | 171 423 |
| TRF E665 | 25 355 | 28 776 | 81 822 |
| B665 | 7380 | 6047 | 5948 |
| Delta 665 | 17 975 | 22 729 | 75 874 |
| H490 | 13 864 | 25 198 | 54 724 |
| Normalized Delta 665 | 12 965 | 9020 | 13 865 |

An increase in both the Hoechst signal (H490) and in the FRET signal is observed, and these signals are proportional to the amount of lysate added, as observed in the previous examples. The TR-FRET signal at 665 nm normalized with respect to the H490 signal gives an average value of approximately 11 000 afu representing the amount of HER1 receptor expressed by the cells of R1R2 type.

### Example 8

### Measurement of the Expression of HER1 (EGFR) Receptors by Measurement of TR-FRET of the Emission of Terbium at 490 nm and of a FRET Acceptor, Compatible with Terbium and Emitting at Around 520 nm, and Normalization by Fluorescence of an Intercalating Agent, Measured at 490 nm or 520 nm in Continuous Fluorescence Mode

### 8.1. Labeling of an Antibody with Alexa-488 as FRET Acceptor

The REGF01 antibody (33 μl at 3 mg/ml in PBS, i.e. 100 μg) was diluted with 66 μl of 0.1 M carbonate buffer, pH 8.5, and 6 μl of a 1.2 mM solution of Alexa-488 (Invitrogen) in DMSO were added. After incubation for one hour at ambient temperature, the reaction mixture was purified on a NAP-5 column equilibrated in 100 mM phosphate buffer, pH 7. A UV spectrum of the excluded fraction (300 μl) shows that the antibody is labeled at a rate of two Alexa-488 molecules per antibody molecule.

8.2.1. An R1R2 cryosection prepared in Example 1 was placed in an eppendorf tube in 360 μl of PBS buffer, pH 7, containing 0.1% of BSA (protease-free quality) and a protease inhibitor (Roche, 1 tablet/10 ml) and 40 μl of a solution of Hoechst 33342 (Invitrogen # H1399 in solution 2 mg/ml DMSO) prediluted to 1/100 in buffer, pH 7, were added. After incubation for 10 min in the dark, the tube was centrifuged and washed with 3 times 400 μl of PBS buffer+0.1% BSA. The pellet formed by the cryosection was taken up in 300 μl of PBS buffer containing 0.1% of BSA and a protease inhibitor (“complete mini” Roche #04 693 124 001; 1 tablet/10 ml). The tube was kept in ice and subjected to sonication (5 s at 20% of the power with a Branson 450 sonicator provided with a “micro tip” probe). 10 μl, 20 μl and 40 μl of the homogeneous suspension obtained were deposited in wells of a microplate (Costar #3694 “96 well half area flat bottom black polystyrene”) and then made up to 80 μl with the same buffer as that used for the sonification step, and 80 μl of the same buffer were deposited in several wells so as to constitute “buffer blanks” (BB).

The REGF08-Lumi4Tb and REGF01-Alexa488 antibodies prepared according to Examples 5.1 and 8.1 were added so as to obtain a final concentration of 1 nM of the REGF08-Lumi4Tb antibody and 2 nM of the REGF01-Alexa488 antibody in the wells; 20 μl of the antibody mixture were also added to 80 μl of buffer so as to obtain a “reagent blank” (RB) (in this case a single antibody concentration was used to calculate the contribution at 520 nm of the donor in the “test” wells).

8.2.2. The plate was then placed on a Pherastar FS instrument provided with excitation via a flashlamp and with a monochromator set at 340 nm (20 nm) for excitation. For the time-resolved (TRF) measurements, the fluorescence was measured at 490 nm after a delay of 60 μs following excitation, and for an integration time of 400 μs; for the continuous fluorescence measurement of Hoechst fluorescence, the fluorescence was measured at 490 nm without delay after excitation, using the “Fluorescence” protocol provided in the software of the instrument.

| | μl of cell suspension | | | |
|---|---|---|---|---|
| | 0 | 10 | 20 | 40 |
| H490 (fluorescence 490 nm after | 8880 | 21 200 | 31 500 | 49 230 |
| Ab addition) | | | | |

It is observed that the fluorescence intensity measured at 490 nm in continuous mode is proportional to the amount of cell suspension deposited in the wells.

| | μl of cell suspension | | | |
|---|---|---|---|---|
| | 0 | 10 | 20 | 40 |
| H520 (fluorescence 520 nm after Ab | 1115 | 12 418 | 22 840 | 40 120 |
| addition | | | | |

The fluorescence was measured at 520 nm using the same plate on the same Pherastar FS instrument without delay after excitation, using the “Fluorescence” protocol provided in the software of the instrument. It is observed that the fluorescence intensity measured at 520 nm in continuous mode is also proportional to the amount of cell suspension deposited in the wells. The measurement of fluorescence of the Hoechst-DNA complex can be measured equally at the wavelength envisioned for the measurement of the fluorescence of the donor or of the acceptor.

The same plate was then measured in TRF mode (delay of 60 μs following excitation, and integration time of 400 μs). In this case, owing to the strong transfer, it is observed that the normalization demonstrates a hook effect. In this case, the variation in fluorescence at 520 nm has a tendency to decrease with the amount of cell suspension added, whereas the H490 fluorescence clearly increases with the amount of cell suspension added.

The results obtained are given in Table 17.

TABLE 17

| | μl of cell suspension | | |
|---|---|---|---|
| | 10 | 20 | 40 |
| E520c | 16 880 | 20 700 | 22 540 |
| E490c | 100 860 | 85 890 | 93 850 |
| B520 | 4822 | 4100 | 4490 |
| Delta520 | 12 060 | 16 600 | 18 060 |
| F490 | 8880 | 21 200 | 31 500 |
| Delta520N | 13 580 | 7830 | 5730 |

### Example 9

### Measurement of the Expression of HER1 (EGFR) Receptors by Measurement of TR-FRET of the Emission of Terbium at 490 nm and of a FRET Acceptor Compatible with Terbium and Normalization by the Fluorescence of a Fluorescent Protein (GFP), Measured at 490 nm, 545 nm or 590 nm in Continuous Fluorescence Mode

### 9.1. Obtaining of a Polyclonal Cell Line Stably Expressing HER1 (EGFR) Receptors and Transient Transfection of a Fluorescent Protein (GFP)

HEK cells (background HEK ATCC) were transfected in the presence of lipofectamine 2000 using a plasmid encoding the HER1 (EGFR) receptor, this plasmid also comprising a Geneticin resistance gene. After transfection, the cells were subjected to selection by adding Geneticin (0.6 mg/ml) to the culture medium so as to select the population of resistant cells having stably integrated the plasmid. The cells (polyclonal population) thus obtained are called HEK-EGFR.

Transient transfection of the HEK-EGFR polyclonal line with GFP (HEK-EGFR-GFP cells): A mixture composed of 8 ml of optiMEM medium+60 μl lipofectamine 2000+20 μg of pmaxGFP plasmid (positive control of the Amaxa Nucleofection kit) preincubated for 20 min at ambient temperature was added to the HEK-EGFR cells in a T175 flask (cells at approximately 70% of confluence, i.e. ˜20 million cells) and then 12 ml of complete medium+0.6 mg/ml Geneticin were added to the cells.

After incubation for 24 h in the incubator (37° C.+CO₂), the cells were washed (PBS), and detached with 5 ml of Cell Dissociation Buffer (Sigma); the cell suspension was then centrifuged and the pellet taken up in Krebs buffer. After counting of the cells on a ViaCell counter, the suspension was diluted with Krebs buffer so as to adjust the concentration, and then the diluted suspensions were distributed into the wells of a 96-well Cellstar black microtitration plate (pretreated with polyornithine) so as to have a known cell density, for example of 25K (=25 000 cells), 50K and 200K HEK-EGFR-GFP cells in a volume of 100 μl per well. “Buffer blanks” are also formed by adding 100 μl of Krebs buffer to adjacent wells.

### 9.2. Measurements of Fluorescence in “Continuous Fluorescence” Mode on HEK-EGFR-GFP Cells

The (continuous mode) fluorescence emission spectra of the wells of a plate comprising series of wells containing respectively 25K, 50K and 200K cells were recorded on a Tecan Saphir 2 instrument with the following parameters, using the preferred wavelength for the excitation of the GFP lying in the near ultra-violet range. Wild-type GFP (wGFP) has two excitation maxima, at 395 nm (UV light) and at 475 nm (blue light) (respective extinction coefficients of 30 000 M⁻¹ cm⁻¹ and 7000 M⁻¹ cm⁻¹):
Excitation 395 nm (20 nm)
Emission (5 nm)
Delay 0 μs
Integration 20 μs
Gain 171

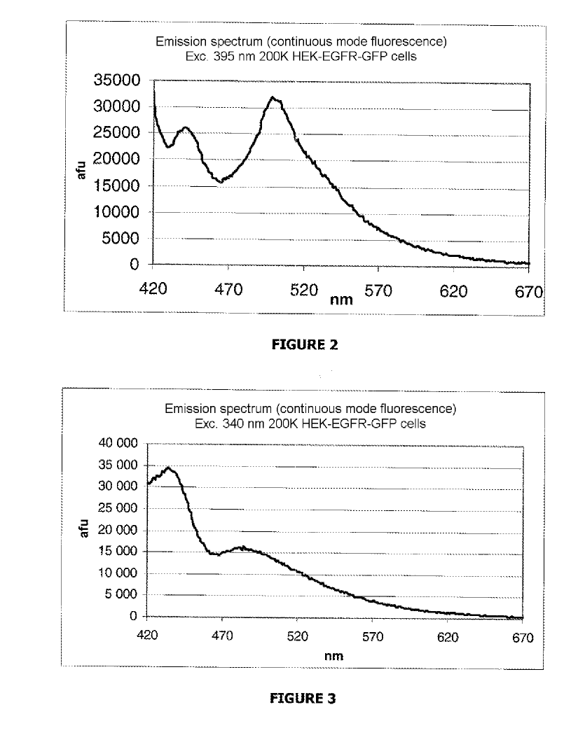

The spectrum obtained shows an emission maximum around 500 nm (FIG. 2).

The (continuous mode) fluorescence emission spectra of the wells of the same plate were recorded on the Tecan Saphir 2 instrument with the following parameters using an excitation wavelength of 340 nm normally used for the excitation of lanthanide complexes without changing the other parameters:
Excitation 340 nm (20 nm)
Emission (5 nm)
Delay 0 μs
Integration 20 μs
Gain 171

The spectrum obtained shows an emission maximum around 500 nm, the intensity of which is approximately half that observed when exciting at 395 nm (FIG. 3).

The fluorescence intensities of the wells of the same plate were measured on the Tecan Saphir 2 instrument:
a) either using the wavelength normally preferred for measuring the fluorescence of GFP:
   Excitation 395 nm (20 nm)
   Emission 509 (10 nm)
   Delay 0 μs
   Integration 20 μs
   Gain 150
b) or using an excitation wavelength of 340 nm normally used for the excitation of lanthanide complexes:
   Excitation 340 nm (20 nm)
   and measuring the fluorescence intensities at the wavelengths corresponding to the main terbium emission lines (490 nm and 545 nm) and also at 509 nm, the wavelength normally used to measure the fluorescence of GFP.
   Emission 490 (10 nm), emission 509 (10 nm), emission 545 (10 nm)
   Delay 0 μs
   Integration 20 μs
   Gain 150

The results (average of 10 measurements) have been grouped together in Table 18.

TABLE 18

| | F 490 | F 509 | F 545 | F 509 |
|---|---|---|---|---|
| ×10³cells | (Ex 340) | (Ex 340) | (Ex 340) | (Ex 395) |
| 0 | 21 387 | 18 331 | 9992 | 16 570 |
| 50 | 22 926 | 21 269 | 11 217 | 34 234 |
| 200 | 29 847 | 25 852 | 13 764 | 54 568 |
| Correlation | R² = 0.995 | R² = 0.9761 | R² = 0.9761 | R² = 0.9433 |
| coefficient | | | | |

This example shows that GFP can be excited at the wavelength of 340 nm in place of the wavelength of 395 nm normally used. Likewise, the fluorescence can be preferentially measured at 490 nm and at 545 nm; this is also possible at 590 nm, but, in this case, and with the cells used in this example, a minimum of 50K cells is necessary in order to have a correct signal/noise ratio.

It was observed that, unexpectedly, the correlation coefficient corresponding to a linear regression indicates a better linearity for the conditions corresponding to an excitation at 340 nm and more particularly for the measurement using the pair [Ex 340 nm; Em 490 nm] corresponding to the excitation conditions used for the measurements of intensities on terbium complexes.

### 9.3. Measurements of the Expression of the EGFR Receptor by TR-FRET and Normalization by “Continuous Fluorescence” Mode Measurement of the Emission of GFP on HEK-EGFR-GFP Cells

10 μl of a solution containing REGF-08-Lumi4Tb antibody (25 nM) and REGF01-Alexa488 antibody (25 nM) in buffer (PBS+0.1% BSA) were added to the previous plate in the wells containing known amounts of HEK-EGFR-GFP cells and also in one of the wells containing Krebs buffer so as to constitute a “reagent blank” (RB). The “reagent blank” makes it possible to calculate the contribution of the terbium in the 520 nm “acceptor” channel and to be able to calculate the variation in fluorescence at 520 nm originating from the FRET and thus to be able to assay the expression of the EGFR receptor.

The continuous fluorescence mode measurement gives the following values, the variation in emission of the acceptor at 520 nm is calculated as in Example 8 by calculating the contribution of the terbium emission in the 520 nm channel (B520), using, as calibrators, wells containing only labeled antibodies in buffer. Delta 520=E520−B520; this value represents the FRET signal corresponding to the measurement of the expression of the EGFR receptor. The value Delta 520N=(Delta 520/F 490)×10 000 corresponds to the value of the expression of the EGFR receptor normalized with respect to the F490c fluorescence measurement (GFP signal).

The plate was read on a Pherastar FS instrument with excitation via flashlamp in fluorescence mode with a 340 nm filter for excitation and 490 and 520 filters for emission.

| Cells | 25K | 50K | 200K |
|---|---|---|---|
| E490 | 15 880 | 23 900 | 49 780 |

| Cells | 25K | 50K | 200K |
|---|---|---|---|
| F520 | 54 000 | 62 190 | 88 170 |

The GFP fluorescence could therefore be measured either at the wavelength of one of the terbium emission lines (490 nm) or at the emission wavelength of the acceptor (520 nm) (itself excited by FRET using terbium). In the two cases, the fluorescence emission is proportional to the number of cells (amount of biological material).

The TRF mode measurements were carried out with the following parameters:
Optical module: 337, 520, 490
Excitation: 337 nm
Delay: 60 μs
Integration: 400 μs

The results are grouped together in Table 19.

TABLE 19

| | TRF520 | | |
|---|---|---|---|
| | Delta 520 | F490c | delta 520 N |
| 25K | 2930 | 15 880 | 1850 |
| 50K | 4720 | 23 900 | 1970 |
| 200K | 11 460 | 49 780 | 2300 |

It was observed that the Delta 520 signal measured in time-resolved mode increases proportionally to the number of cells contained in each well, but that Delta 520N representing the expression of the receptor (normalized by the fluorescence measurement measured at 490 nm in continuous fluorescence mode) is constant, on average 2040 afu. Thus, it is possible to use the fluorescence of a protein such as GFP to normalize a TR-FRET signal.

### Example 10

### Labeling of Cells with Dansyl Chloride

Polyclonal cells stably expressing EGFR (see Example 9) were used. Approximately 2000 K cells (2×10⁶ cells) in suspension in 1 ml of Krebs buffer in a 1.5 ml eppendorf were centrifuged for 10 min at a relative centrifugal force (RCF) of 8000, then washed with 3 times 300 μl of 0.1M NaHCO₃ buffer, pH 9, and then resuspended in 150 μl of NaHCO₃ buffer. A 5 mM solution of dansyl chloride in a mixture (3/2) of dioxane and of 0.1 M NaHCO₃ buffer was prepared, and then 3 μl of this solution were added to the cell suspension. After incubation for 30 min with mechanical stirring, the tube was centrifuged for 10 min at an RCF of 8000, the supernatant was removed, and then the pellet was resuspended in 300 μl of carbonate buffer, then centrifuged for 10 min; this operation was repeated once again with 300 μl of carbonate buffer and then once with 300 μl of PBS.

The pellet was resuspended in 500 μl of PBS buffer containing 0.2% of BSA and a protease inhibitor (Roche, 1 tablet/10 ml). The tube is cooled in ice and subjected to sonication (5 s at 20% of the power) and then 10, 20, 40 and 80 μl of the suspension are deposited in the wells of a microplate, the volume being made up to 80 μl with the same buffer.

The preparation of the cells labeled with the dansyl group was based on the publication by Y. Uratani Journal of Bacteriology 1982 (p. 523-528).

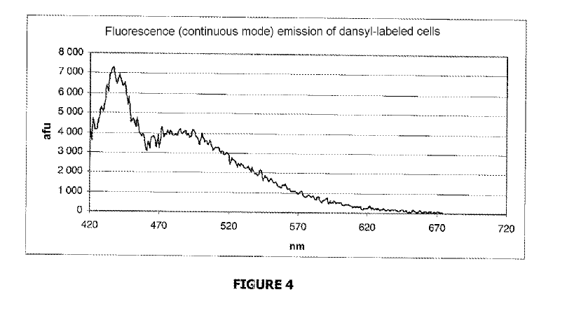

An emission spectrum recorded on a Tecan Saphir II instrument with the following parameters showed (FIG. 4) a maximum fluorescence emission around 500 nm corresponding to the fluorescence data known for dansyl derivatives (excitation 330 nm, emission 510 nm).
Excitation 340 nm (20 nm)
Emission bandwidth: 5 nm
Gain: 158
Integration: 20 μs
Delay: 0 μs

The (continuous mode) fluorescence was also measured at fixed wavelengths corresponding to terbium or europium emission lines (490 nm, 545 nm, 590 nm, 620 nm) and also at 520 nm corresponding to the emission wavelength of an acceptor such as Alexa-488 or fluorescein using the following parameters:
Excitation: 340 nm (bandwidth: 20 nm)
Emission bandwidth: 10 nm
Delay: 0 μs
Integration: 20 μs

The results are grouped together in Table 20.

TABLE 20

| | μl of cell suspension | | | | |
|---|---|---|---|---|---|
| | 10 | 20 | 40 | 80 | R² |
| F490 | 2740 | 4730 | 10 820 | 30 910 | 0.9783 |
| F520 | 2640 | 4620 | 9620 | 25 500 | 0.9835 |
| F545 | 3970 | 6800 | 12 930 | 33 850 | 0.9821 |
| F590 | 3100 | 5620 | 11 190 | 30 330 | 0.9813 |
| F620 | 1600 | 2480 | 4190 | 12 210 | 0.9639 |

It was observed that, at the various emission wavelengths tested, the fluorescence is proportional to the amount of biological material, labeled with dansyl chloride, present in the wells.

Measurement of the Expression of EGFR Receptors by HTRF on Cell Lysate after Labeling with Dansyl Chloride

The labeled anti-EGFR antibodies REGF08-Lumi4Tb and REGF01-d2 prepared according to Example 5.1 were added to the wells of a microplate containing 10 μl, 20 μl and 40 μl of cell lysate obtained after labeling with dansyl chloride, so as to obtain a final concentration in the wells respectively of 2.7 nM (REGF08-Lumi4Tb) and 7.3 nM (REGF01-d2). A well containing only the labeled antibodies (“reagent blank”) was used to measure the contribution of the terbium cryptate at 665 nm (B 665) which was used for the calculation of the variation in the TRF 665 signal (Delta 665) due to the FRET. A fluorescence reading was carried out on a PheraStar FS instrument with excitation via a flashlamp and using the following parameters:
Fluorescence mode (continuous):
   Excitation 337 nm
   Emission 490 nm
TRF mode:
   Excitation 337 nm
   Emission A 665 nm
   Emission B 490 nm
   Integration 60 μs
   Integration 400 μs
   Flashlamp

The results are grouped together in Table 21.

TABLE 21

| | Cell lysate (μl) | | |
|---|---|---|---|
| | 10 | 20 | 40 |
| TRF 490c | 550 900 | 524 100 | 540 770 |
| TRF 665c | 19 330 | 19 220 | 20 470 |
| B 665 | 18 400 | 17 500 | 18 060 |
| Delta 665 | 930 | 1720 | 2400 |
| F490 | 1290 | 1980 | 3590 |
| Delta 665N | 724 | 870 | 670 |

It was observed that the variation in fluorescence in time-resolved mode (Delta 665) increases with the amount of biological material introduced into the wells. The Delta 665 normalized by the fluorescence measurement at 490 nm (Delta 665N) represents the expression of the vector; this value, on average 750 afu, is independent of the amount of cell material.

### Example 11

### Comparison Between the Hoechst 33342 Signal Measured According to the Invention and that Obtained Using a Reference Method for Protein Quantification (Sigma Quantipro™ Kit)

Cryosections of tissues (tumor), 50 μm thick, in 200 μL of PBS buffer (without the addition of BSA) containing a protease inhibitor (Roche, 1 tablet/10 ml) were labeled with Hoechst 33342 (20 μL of a solution at 100 μg/mL PBS). After incubation for 10 min, the tube was centrifuged (3 min at 12 000 rpm) and the pellet was washed with twice 200 μl of PBS buffer (without the addition of BSA) containing a protease inhibitor; the pellet taken up with 300 μl of the same buffer was cooled in ice and then subjected to sonication, under the same conditions as those of Example 7.

A volume of 90 μl of each homogenate was diluted by half in PBS buffer and then the total protein concentration was measured using the Sigma Quantipro™ BCA kit, in the 96-well microplate format, according to the protocol of the kit. The absorbence values were converted into concentration of total proteins expressed in μg/ml using a calibration line obtained with a BSA concentration range. The total protein concentrations measured are reported in Table 22.

In parallel, 50 μL of each homogenate were deposited (duplicates) in the wells of a microtitration plate (Corning® “black 96 well half-area”) and the fluorescence reading was carried out on an instrument of Pherastar FS type provided with excitation via a flashlamp, with a 337 nm excitation filter and with a 490 nm emission filter (HTRF optical modules). The time-resolved (TRF) measurements were carried out in decay curve mode allowing the acquisition of the fluorescence in “steps” of 2 μs. For the measurement of Hoechst 33342 fluorescence, the fluorescence was measured at 490 nm in a range of 38 to 42 μs by summation of the fluorescence of steps 38 to 42 (Raw Data 490 B obtained in Excel table form by the MARS software allowing exportation of the data obtained on the Pherastar instrument). These fluorescence measurements (F490 {38-42} are reported in Table 22.

TABLE 22

| | Sample | | | | | | |
|---|---|---|---|---|---|---|---|
| | B2 | B7 | B32 | B43 | B69 | B76 | B88 |
| Protein μg/ml | 84 | 142 | 166 | 67 | 368 | 264 | 326 |
| F490 {38-42} | 67 590 | 80 800 | 81 640 | 37 050 | 117 500 | 114 500 | 126 340 |

The Hoechst signal measured under the conditions of the invention correlates well with that obtained using a conventional method for protein quantification (R²=0.847). The two methods can therefore be used satisfactorily to estimate the amount of biological material present in a sample, but the normalization according to the invention has the additional advantage of not destroying the sample, whereas the Quantipro® kit requires the destruction of a third of this sample.

1. A process for measuring the luminescence of a long-life fluorescent compound present in a measuring medium, said medium containing a biological sample, which comprises the following steps:
a) introduction of a long-life fluorescent compound into the measuring medium,
b) introduction, into the measuring medium, of a fluorescent labeling agent, the absorption spectrum of which allows the excitation thereof at the same wavelength as that used to excite the long-life fluorescent compound, and the emission spectrum of which allows the measurement of the luminescence thereof at the same wavelength as that used to measure the luminescence of the long-life fluorescent compound,
c) excitation of the measuring medium at a wavelength corresponding to an absorption peak of the long-life fluorescent compound,
d) measurement of the luminescence emitted by the measuring medium immediately after the excitation of said medium, mainly corresponding to the luminescence of the labeling agent, and for a period of 5 ns to 45 μs, at a wavelength corresponding to an emission peak of the long-life fluorescent compound,
e) time-resolved measurement of the luminescence emitted by the measuring medium at the same wavelength as that used in step d), after a delay of 20 to 200 μs following the excitation of the measuring medium and for a period of 200 to 1000 μs, this luminescence mainly corresponding to that of the long-life fluorescent compound,
f) calculation of a normalized luminescence signal corresponding to the ratio: (signal obtained in step e)/(signal obtained in step d).

2. A process for measuring the luminescence of a long-life fluorescent compound present in a measuring medium, said medium containing a biological sample, which comprises the following steps:
a) introduction of a long-life fluorescent compound into the measuring medium,
b) introduction, into the measuring medium, of an acceptor fluorescent compound, the absorption spectrum of which is compatible with the emission spectrum of the long-life fluorescent compound, the long-life fluorescent compound and the acceptor fluorescent compound being FRET partners,
c) introduction, into the measuring medium, of a fluorescent labeling agent, the absorption spectrum of which allows the excitation thereof at the same wavelength as that used to excite the long-life fluorescent compound, and the emission spectrum of which allows the measurement of the luminescence thereof at the same wavelength as that used to measure the luminescence of the long-life fluorescent compound,
d) excitation of the measuring medium at a wavelength corresponding to an absorption peak of the long-life fluorescent compound,
e) measurement of the luminescence emitted by the measuring medium immediately after the excitation of said medium, mainly corresponding to the luminescence of the labeling agent, and for a period of 5 ns to 45 μs, at a wavelength corresponding to an emission peak of the long-life fluorescent compound,
f) optionally, time-resolved measurement of the luminescence emitted by the measuring medium at the same wavelength as that used in step e), after a delay of 20 to 200 μs following the excitation of the measuring medium and for a period of 200 to 1000 μs,
g) time-resolved measurement of the luminescence emitted by the measuring medium after a delay of 20 to 200 μs after the excitation of the measuring medium and for a period of 200 to 600 μs, at the emission wavelength of the acceptor compound,
h) determination of a normalized TR-FRET signal comprising the calculation of the ratio: (signal obtained in step g)/[(optionally signal obtained in step f)×(signal obtained in step e)].

3. The process as claimed in claim 1, wherein:
the long-life fluorescent compound is a europium cryptate or chelate;
the fluorescent labeling agent is an agent the absorption spectrum of which allows excitation at a wavelength of between 330 and 350 nm and which emits at the wavelengths of 588 nm+/−10 nm or 620 nm+/−10 nm;
the luminescence of the long-life fluorescent compound and that of the fluorescent labeling agent are both measured at the wavelength of 588 nm+/−10 nm or 620 nm+/−10 nm.

4. The process as claimed in claim 1, wherein:
the long-life fluorescent compound is a terbium cryptate or chelate;
the fluorescent labeling agent is an agent the absorption spectrum of which allows excitation at a wavelength of between 330 and 350 nm and which emits in particular at the wavelengths of 490 nm+/−10 nm, 545 nm+/−10 nm or 590+/−10 nm;
the luminescence of the long-life fluorescent compound and that of the fluorescent labeling agent are both measured at the wavelength of 490 nm+/−10 nm, 545 nm+/−10 nm or 590+/−10 nm.

5. The process as claimed in claim 2, wherein:
in step c), the labeling agent is a fluorescent labeling agent, the absorption spectrum of which allows the excitation thereof at the same wavelength as that used to excite the long-life fluorescent compound, and the emission spectrum allows the measurement of the luminescence thereof at the same wavelength as that used to measure the luminescence of the acceptor fluorescent compound,
in step e), the luminescence is measured at a wavelength corresponding to an emission peak of the acceptor fluorescent compound,
in step f), if it is present, the luminescence is measured at a wavelength corresponding to an emission peak of the long-life fluorescent compound.

6. The process as claimed in claim 5, wherein:
the long-life fluorescent compound is a europium or terbium cryptate or chelate,
the fluorescent labeling agent is an agent the absorption spectrum of which allows excitation at a wavelength of between 330 and 350 nm and which emits a wavelength of between 450 and 650 nm, and
the luminescence of the acceptor fluorescent compound and that of the fluorescent labeling agent are both measured at a wavelength included in these ranges.

7. The process as claimed in claim 1, which comprises a second step of excitation of the measuring medium at a wavelength corresponding to an absorption peak of said long-life fluorescent compound, said second excitation being carried out immediately before the first step of time-resolved measurement of the luminescence.

8. The process as claimed in claim 1, wherein the biological sample is chosen from: a tissue extract; a tumor sample; and cells derived from a cell culture.

9. The process as claimed in claim 1, wherein the biological sample has been the subject of a treatment aimed at homogenizing said sample in cell lysate form, before the introduction of the fluorescent compounds into the measuring medium.

10. The process as claimed in claim 1, wherein the biological sample has been the subject of a treatment aimed at homogenizing said sample in cell lysate form, after the introduction of the fluorescent compounds into the measuring medium.

11. The process as claimed in claim 1, wherein the long-life fluorescent compound is a fluorescent metallic complex chosen from: a europium cryptate; a europium chelate; a terbium chelate; a terbium cryptate; a ruthenium chelate; and a quantum dye.

12. The process as claimed in claim 1, wherein the labeling agent is chosen from: a fluorescent intercalating agent, a fluorescent protein, a fluorescent compound which labels mitochondria, a fluorescent compound which binds to the amine functions of proteins, and a fluorescent compound which accumulates in lipid membranes.

13. The process as claimed in claim 1, wherein the labeling agent is a fluorescent intercalating agent.

14. The process as claimed in claim 13, wherein the fluorescent intercalating agent is chosen from the following compounds: Hoechst 33258; Hoechst 33342; Hoechst 34580; and DAPI (4′,6′-diamidino-2-phenylindole).

15. The process as claimed in claim 1, wherein the acceptor fluorescent compound, when it is present, is chosen from: allophycocyanins; luminescent organic molecules, squaraines, coumarins, proflavines, acridines, fluoresceins, boron-dipyrromethene derivatives; fluorophores known under the name “Atto”; fluorophores known under the name “DY”; compounds known under the name “Alexa”; nitrobenzoxadiazole; and fluorescent proteins.

16. A reagent kit comprising:
a long-life fluorescent compound;
a fluorescent labeling agent, the absorption spectrum of which allows the excitation thereof at the same wavelength as that used to excite the long-life fluorescent compound, and the emission spectrum of which allows the measurement of the luminescence thereof at the same wavelength as that used to measure the luminescence of the long-life fluorescent compound;
optionally, an acceptor fluorescent compound, the absorption spectrum of which is compatible with the emission spectrum of the long-life fluorescent compound, the long-life fluorescent compound and the acceptor fluorescent compound being FRET partners.

17. The reagent kit as claimed in claim 16, wherein:
the long-life fluorescent compound is chosen from: a europium cryptate; a europium chelate; a terbium chelate; and a terbium cryptate;
the fluorescent labeling agent has an absorption spectrum which allows the excitation thereof between 330 and 350 nm and an emission spectrum which allows the measurement of the luminescence thereof at the same wavelength as that used to measure the luminescence of the long-life fluorescent compound.

18. A reagent kit comprising:
a long-life fluorescent compound;
an acceptor fluorescent compound, the absorption spectrum of which is compatible with the emission spectrum of the long-life fluorescent compound, the long-life fluorescent compound and the acceptor fluorescent compound being FRET partners;
a fluorescent labeling agent, the absorption spectrum of which allows the excitation thereof at the same wavelength as that used to excite the long-life fluorescent compound, and the emission spectrum of which allows the measurement of the luminescence thereof at the same wavelength as that used to measure the luminescence of the acceptor fluorescent compound.

19. The reagent kit as claimed in claim 18, wherein:
the long-life fluorescent compound is chosen from: a europium cryptate; a europium chelate; a terbium chelate, and a terbium cryptate;
the fluorescent labeling agent has an absorption spectrum which allows the excitation thereof between 330 and 350 nm and an emission spectrum which allows the measurement of the luminescence thereof at the same wavelength as that used to measure the luminescence of the acceptor fluorescent compound.

20. The kit as claimed in claim 16, wherein the labeling agent is chosen from: a fluorescent intercalating agent, a fluorescent protein, a fluorescent compound which labels mitochondria, a fluorescent compound which binds to the amine functions of proteins, and a fluorescent compound which accumulates in lipid membranes.

21. The kit as claimed in claim 20, wherein the labeling agent is a fluorescent intercalating agent.

22. The kit as claimed in claim 21, wherein the fluorescent intercalating agent is chosen from the following compounds: Hoechst 33258; Hoechst 33342; Hoechst 34580; and DAPI (4′,6′-diamidino-2-phenylindole).

23. The process as claimed in claim 6, wherein the fluorescent labeling agent emits a wavelength of between 490 and 600 nm.

24. The process as claimed in claim 8, wherein the biological sample is a histological section.

25. The kit as claimed in claim 18, wherein the labeling agent is chosen from: a fluorescent intercalating agent, a fluorescent protein, a fluorescent compound which labels mitochondria, a fluorescent compound which binds to the amine functions of proteins, and a fluorescent compound which accumulates in lipid membranes.

26. The kit as claimed in claim 25, wherein the labeling agent is a fluorescent intercalating agent.

27. The kit as claimed in claim 26, wherein the fluorescent intercalating agent is chosen from the following compounds: Hoechst 33258; Hoechst 33342; Hoechst 34580; and DAPI (4′,6′-diamidino-2-phenylindole).
